(19) United States
(12) Patent Application Publication
(10) Pub. No.: US 2022/0184172 A1
(43) Pub. Date: Jun. 16, 2022

(54) AMPK/CASPASE-6 AXIS CONTROLS LIVER DAMAGE IN NONALCOHOLIC STEATOHEPATITIS

(71) Applicant: The Regents of the University of California, Oakland, CA (US)

(72) Inventors: Alan Salitiel, La Jolla, CA (US); Peng Zhao, La Jolla, CA (US)

(73) Assignee: The Regents of the University of California, Oakland, CA (US)

(21) Appl. No.: 17/602,966

(22) PCT Filed: Apr. 18, 2020
(86) PCT No.: PCT/US2020/028878

§ 371 Date: Oct. 11, 2021

(30) Foreign Application Priority Data
Apr. 19, 2019 (US) 62836183

(51) Int. Cl. A61K 38/07; A61K 31/4365; A61K 45/06; A61P 1/16; A61P 43/00

(52) U.S. Cl. CPC A61K 38/07; A61K 31/4365; A61P 43/00; A61P 1/16; A61K 45/06

(57) ABSTRACT

The present disclosure provides a method of preventing and/or treating hepatocellular apoptosis and liver damage in a liver disease, particularly NASH, by targeting the AMPK/caspase-6 axis to inhibit caspase-6 activity and/or activate AMPK activity. Also disclosed is the pharmaceutical composition for preventing and/or treating a liver disease comprising one or more caspase-6 inhibitor and/or AMPK activator of the present disclosure. This abstract is intended as a scanning tool for purposes of searching in the particular art and is not intended to be limiting of the present disclosure.

### CROSS-REFERENCE TO RELATED APPLICATION

This PCT International Application claims benefit and priority to U.S. Provisional Application No. 62/836,183, filed on Apr. 19, 2019, the entire content of which is hereby incorporated by reference.

### STATEMENT OF GOVERNMENT SUPPORT

This invention was made with government support under DK063491, DK076906, DK117551, and HL143277awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 16, 2020, is named 942103_2010_PCT_SL.txt and is 19,051 bytes in size.

### FIELD OF INVENTION

The present disclosure relates generally to a method of preventing and/or treating liver disease, particularly nonalcoholic steatohepatitis (NASH).

### BACKGROUND OF INVENTION

Nonalcoholic steatohepatitis (NASH), characterized by hepatic steatosis, inflammation and liver damage, has become a leading cause of liver transplant and liver-associated death. Hepatocellular death, characterized by swollen hepatocytes on liver biopsy, is a cardinal feature of NASH (1, 2). In healthy liver, hepatocyte apoptosis has a key role in liver homeostasis, maintaining equilibrium between hepatocyte loss and replacement (3). However, pathological conditions such as viral infection, alcoholic or nonalcoholic steatohepatitis, and physical injury lead to extensive hepatocyte apoptosis and liver damage (4), which cause progressive fibrosis and cirrhosis (1, 5). Improving liver damage and preventing fibrosis are major goals of NASH therapy (2). Moreover, liver cell death is a major contributor to the pathogenesis of hepatocellular carcinoma (2). Therefore, understanding the molecular mechanisms controlling hepatocellular death may lead to new treatments for liver diseases.

AMP-activated protein kinase (AMPK) is a key metabolic regulator that senses energy status and controls energy expenditure and storage (6). AMPK is allosterically activated by AMP and repressed by ATP (6). Its activity is increased during undernutrition (7) and decreased during obesity (8, 9), hyperglycemia(9), and by inhibitory phosphorylation driven by hyperinsulinemia and inflammation (10-12). Although activation of hepatic AMPK attenuates high fat diet (HFD)-induced nonalcoholic fatty liver (NAFL), reducing AMPK activity does not cause or further worsen it (13). Whether the pathogenic repression of AMPK activity in obesity contributes to the occurrence of NASH and NASH-associated liver damage remains unknown.

Caspases are related aspartic-serine proteases that regulate inflammation and cell death. Apoptotic caspases are classified as “initiator”, such as caspase-8 and -9, or “executioner”, including caspase-3, -6, and -7 (14). Apoptotic cell death occurs through extrinsic and intrinsic pathways (15). The extrinsic pathway is driven by extracellular death receptor ligands, such as the Tumor Necrosis Factor (TNF) superfamily and Fas ligand and mediated by caspase-8. The intrinsic pathway is triggered by intracellular stress-induced cytochrome c release from mitochondria, leading to activation of the Apaf1-caspase-9 apoptosome. Both pathways converge in cleavage and activation of caspase-3 and -7 to execute programmed cell death (15). Although classified as an executioner, the mechanisms of activation and cleavage, and the function of caspase-6 remain uncertain (14). It is found that caspase-6 functions in steatosis-induced hepatocyte death, and integrates signals from both inflammation and energy metabolism through direct phosphorylation by AMPK. Steatosis-induced decline in AMPK-catalyzed phosphorylation permits caspase-6 activation, leading to hepatocyte death. This link to obesity suggests that the AMPK-caspase-6 axis has a key role in NASH and might represent a new therapy.

### SUMMARY OF THE INVENTION

The present disclosure provides that the AMPK/caspase-6 axis plays a key role in the development of NASH and represents a new site for therapeutic intervention. Accordingly, disclosed herein is a method of treating and/or preventing liver disease in a patient in need thereof, by targeting the AMPK/caspase-6 axis to inhibit caspase-6 activity and/or to activate AMPK activity. The liver disease can be any liver disease, including, but not limited to, chronic and/or metabolic liver diseases, nonalcoholic fatty liver disease (NAFLD), and nonalcoholic steatohepatitis (NASH).

In certain embodiments, provided herein is a method of treating and/or preventing nonalcoholic steatohepatitis (NASH) in a patient in need thereof, comprising administering to the patient a therapeutically effective amount of an inhibitor, such as the caspase-6 inhibitor Z-VEID-FMK (“VEID” disclosed as SEQ ID NO: 71), that inhibits caspase-6 activity. In certain embodiments, provided herein is a method of treating and/or preventing nonalcoholic steatohepatitis (NASH) in a patient in need thereof, comprising administering to the patient a therapeutically effective amount of an activator, such as the AMPK agonist A-769662, that activates AMPK activity. The present disclosure encompasses any caspase-6 inhibitors and/or AMPK activators/agonists, now known and/or later developed, that are capable of targeting the AMPK/caspase-6 axis.

In the methods provided herein, the therapeutic agent that targets the AMPK/caspase-6 axis can be a single and/or a combination of at least one such caspase-6 inhibitor and/or AMPK activator. In certain embodiments, the therapeutic agent of the present disclosure can be administered in a single pharmaceutical composition, or separately in more than one pharmaceutical composition. Accordingly, also provided herein is a pharmaceutical composition comprising a therapeutically effective amount of single and/or a combination of at least one therapeutic agent of the present disclosure that targets the AMPK/caspase-6 axis to inhibit caspase-6 activity and/or activate AMPK activity.

Also disclosed, in various embodiments herein, are uses of a disclosed therapeutic agent targeting the AMPK/caspase-6 axis, or a pharmaceutically acceptable salt thereof, in the manufacture of a medicament for the treating and/or preventing liver disease in a patient in need thereof.

### BRIEF DESCRITION OF THE DRAWINGS

Many aspects of the present disclosure can be better understood with reference to the following drawings.

FIGS. 1A-1P. Liver-specific AMPK knockout exaggerates liver damage in NASH.

FIG. 1A. Expression of Prkaa1 and Prkaa2 in liver. n=8-9. FIG. 1B Immunoblot (IB) of liver lysate from ND or CD-HFD-fed mice. n=3. (C-P) Flox and LAKO mice fed CD-HFD for 11 weeks. FIG. 1C. Body weight. FIG. 1D. liver weight. FIG. 1E. Liver triglyceride. FIGS. 1F-1H. Serum ALT (FIG. 1F), AST (FIG. 1G), ALP (FIG. 1H). n=8-9. FIG. 1I. Liver sections stained as indicated: TUNEL, DAPI, or pMLKL. In the merged image section, the different stains are as indicated by differing shades of grayscale. Scale bar=50 μm. FIG. 1J. Quantification of TUNEL-positive nuclei per field in (FIG. 1I). n=7. FIG. 1K. H&E, F4/80 and Sirius red staining of liver sections. Scale bar=100 μm. n=7. FIG. 1L. Quantification of fibrosis area (% of total area) shown in (FIG. 1K). n=7. FIG. 1M. Liver hydroxyproline. n=8-9. FIG. 1N. Expression of Tnfα, Ccl2, Ccr2, Il1b and Adgre1 in liver. n=8-9. FIG. 1O. Expression of Casp3, Casp8, Ripk1 and Ripk3 in liver. n=8-9. FIG. 1P. Expression of Tgfb, Timp1, Col1a1, Col3a1, Acta2, Pdgfa, Pdgfb, Pdgfra and Ddr2 in liver. n=8-9. Mean±SEM. *, P<0.05, Student's unpaired t test.

FIGS. 2A-2H. AMPK deficiency increases caspase-6 cleavage to promote liver damage in NASH. FIG. 2A-2E. Flox and LAKO mice fed CD-HFD for 11 weeks. FIG. 2A. IB analysis of liver lysate. n=6. FIG. 2B. Casp6 activity in liver lysate. n=7-8. FIG. 2C. Liver sections stained as indicated: active Caspase-6 and DAPI. In the merged image section, the different stains correspond to the grayscale shades as indicated in the single stained images to the left. Scale bar=50 μm. FIG. 2D. Quantification of aCasp6 staining in FIG. 2C. n=7. FIG. 2E. Liver sections stained as indicated: TUNEL, aCasp6 and DAPI. In the merged image section, the different stains correspond to the grayscale shades as indicated in the single stained images above the merged image. Scale bar=50 μm. *, P<0.05, Student's unpaired t test. FIG. 2F-2H. Flox and LAKO mice fed with CD-HFD for 3 weeks, followed by intravenously injection of 1.5 mg/kg caspase-6 siRNA (KD) or scrambled RNA (Sc) twice per week for 3 weeks while continuous CD-HFD. FIG. 2F. Liver sections stained as indicated: TUNEL and DAPI. In the merged image section, the different stains correspond to the grayscale shades as indicated in the single stained images to the left. Scale bar=50 μm. (G) Quantification of TUNEL-positive nuclei per field in FIG. 2F. n=7. FIG. 2H. Serum ALT. n=7-10. Mean±SEM. *, P<0.05, two-way ANOVA.

FIGS. 3A-3E. Caspase-6 is activated in murine and human NASH. FIG. 3A. Healthy Model: 24 weeks old male C57BL/6J mice fed ND; STAM-NASH model: male C57BL/6J mice were subcutaneously injected 200 μg streptozotocin (STZ) within 48 hours after birth and fed HFD for 6 weeks starting at 4 weeks of age; MUP-uPA-NASH model: male MUP-uPA mice fed 60% HFD for 16 weeks; CD-HFD-NASH Model: C57BL/6J mice were fed CD-HFD for 11 weeks; AMLN-NASH Model: C57BL/6J mice fed AMLN diet for 30 weeks. Liver sections were stained as indicated (aCasp6 and DAPI); in the merged image sections, the different stains correspond to the grayscale shades as indicated in the single stained images to the left or as indicated therein. Scale bar=50 μm. FIG. 3B. Human liver sections were classified blindly by liver pathologist and stained with aCasp6 (Kleiner fibrosis score 0, 1-2 and 3-4). Scale bar=50 μm. FIG. 3C. Quantification of aCasp6 staining in FIG. 3B, plotted against Kleiner fibrosis scores. n=4. FIG. 3D. Human liver sections were stained aCasp6 to compare caspase-6 activation in healthy and cirrhotic donors. Scale bar=50 μm. FIG. 3E. Scanning of human liver sections in FIGS. 3B, 3D, and 3E stained as indicated (aCasp6, TUNEL, and DAPI); in the merged image sections, the different stains correspond to the grayscale shades as indicated in the single stained images to the left or as indicated therein. Scale bar=2 mm. Mean±SEM, *, P<0.05, Student's unpaired t test.

FIGS. 4A-4L. Both an AMPK agonist and a caspase-6 inhibitor therapeutically improve liver damage. FIG. 4A-4I. C57BL/6J mice were fed CD-HFD for 6 weeks, followed by intraperitoneally injection of 25 mg/kg A-769662 or vehicle daily for 2 weeks while continuous CD-HFD. FIG. 4A. Liver sections stained as indicated: TUNEL and DAPI. In the merged image section, the different stains correspond to the grayscale shades as indicated in the single stained images to the left. Scale bar=50 μm. FIG. 4B. Quantification of TUNEL-positive nuclei per field in FIG. 4A. n=7. FIG. 4C-4E. Serum ALT (FIG. 4C), AST (FIG. 4D) and ALP (FIG. 4E). n=7. FIG. 4F. H&E and Sirius red staining of liver sections as indicated. Scale bar=100 μm. FIG. 4G. Quantification of liver fibrosis area (% of total area) in FIG. 4F. n=7. FIG. 4H. Liver hydroxyproline. n=8. FIG. 4I. Expression of Tgfb, Timp1, Col1a1, Col3a1, Pdgfa, Pdgfb, Pdgfra and Ddr2 in livers. n=8. *, P<0.05, Student's unpaired t test. FIGS. 4J-4L. Flox and LAKO mice were fed CD-HFD for 6 weeks, followed by intraperitoneally injection of 5 mg/kg VEID (SEQ ID NO: 71) or vehicle every other day for 2 weeks while continuous feeding. FIG. 4J. Liver sections stained as indicated (TUNEL and DAPI); in the merged image sections, the different stains correspond to the grayscale shades as indicated in the single stained images to the left. Scale bar=50 μm. FIG. 4K. Quantification of TUNEL-positive nuclei per field in FIG. 4J. n=5-6. FIG. 4L. Serum ALT. n=5-6. Mean±SEM. *, P<0.05, two-way ANOVA. FIG. 4 discloses “VEID” as SEQ ID NO: 71.

FIGS. 5A-5K. AMPK phosphorylates caspase-6 to inhibit its cleavage and activation. FIG. 5A. Primary hepatocytes were pretreated 40 μM A-769662 for 1 hr, then treated 30 μg/ml CHX and 50 ng/ml TNFα for 2 hrs. IB analysis of cell lysates. n=3. Mean±SD. *, P<0.05, two-way ANOVA. FIG. 5B. Primary hepatocytes were pretreated 40 μM A-769662 for 1 hr, then treated 250 μM BSA-conjugated palmitic acid (PA) for 2 hrs. Cell lysates were subject to caspase-6 activity assay. Mean±SD. *, P<0.05. FIG. 5C. Caspase-6 Ser²⁵⁷ locates within AMPK substrate motif. FIG. 5C. discloses SEQ ID NOS 57-63, respectively, in order of appearance. FIG. 5D. In vitro kinase assay using recombinant caspase-6, and recombinant AMPKα1β1γ1 or AMPKα2β1γ1 active kinase. FIG. 5E. Alignment of caspase-6 sequence. FIG. 5E. discloses SEQ ID NOS 64-67, respectively, in order of appearance. FIG. 5F. HEK293T cells overexpressing caspase-6-myc WT, S²⁵⁷A, S²⁵⁷D or S²⁵⁷E mutant were treated with 10 μg/ml CHX and 25 ng/ml TNFα for 2 hrs. IB analysis of cell lysates. FIGS. 5G-5J. C57BL/6J mice were fed CD-HFD for 6 weeks, followed by intraperitoneally injection of 25 mg/kg A-769662 or vehicle daily for 2 weeks while continuous CD-HI-D. Mice were sacrificed 6 hrs after last injection. FIG. 5G. IB analysis of liver lysates. n=5. FIG. 5H. Liver lysates were subject to caspase-6 activity assay. n=7. FIG. 5I. Liver sections stained as indicated therein (aCasp6 and DAPI); in the merged image sections, the different stains correspond to the grayscale shades as indicated in the single stained images to the left. Scale bar=50 μm. FIG. 5J. Quantification of aCasp6 staining per field in FIG. 5I. n=7. Mean±SEM. *, P<0.05, Student's unpaired t test. FIG. 5K. IB analysis of liver lysates from C57BL/6J mice fed ND or CD-HFD. Note: AMPK and pAMPK blots are the same as in FIG. 1B.

FIGS. 6A-6I. Caspase-6 mediates a feedforward loop to sustain the caspase cascade. FIG. 6A. In vitro cleavage assay using recombinant procaspase-6 with active caspase-3, -7, -8 or -9. FL, full-length; ΔN, N-terminus deleted form; LG, large; SM, small. FIG. 6B. Primary hepatocytes were pretreated 10 μM caspase-3/7 inhibitor I for 1 hr, then treated 30 μg/ml CHX and 50 ng/ml TNFα for 2 hrs. IB analysis of cell lysates. FIG. 6C. In vitro cleavage assay using purified Bid-HA or Bax-HA expressed in HEK293T cells, and active caspase-6. FIG. 6D. In vitro cleavage assay using recombinant Bid with active caspase-6 or -8. FIG. 6E. In vitro cleavage assay using recombinant Bid with active caspase-6. Bands for cleaved Bid were subject to Edman Degradation. FIG. 6E. discloses SEQ ID NOS 68-69, respectively, in order of appearance. FIG. 6F. Bid sequence and sites cleaved by active caspase-6. FIG. 6F. discloses SEQ ID NO: 70. FIG. 6G. Flox and LAKO mice were fed CD-HFD for 6 weeks, followed by intraperitoneally injection of 5 mg/kg VEID (SEQ ID NO: 71) or vehicle every other day for 2 weeks while continuous CD-HFD. Livers were fractionated to separate cytosolic and mitochondrial extract for IB analysis. FIG. 6H. HepG2 cells transfected scrambled RNA or Caspase-6 siRNA were treated vehicle or 30 μg/ml CHX and 50 ng/ml TNFα for 2 hrs. Medium was changed to remove treatment for 5 hrs. Cell lysates were subject to IB analysis. n=3. Mean±SD. *, P<0.05, two-way ANOVA. FIG. 6I. Proposed model for roles of AMPK-caspase-6 axis in apoptotic caspase cascade.

FIGS. 7A-7G. Liver-specific AMPK knockout does not induce hepatic steatosis or liver damage in ND-fed mice. Flox and LAKO mice were fed with chow diet for 16 weeks. FIGS. 7A-7B. Body weight (FIG. 7A) and liver weight (FIG. 7B) of indicated mice. n=5-6. FIG. 7C. Measurement of liver triglyceride (TG) of indicated mice. n=5-6. FIGS. 7D-7F. Serum ALT (FIG. 7D), AST (FIG. 7E) and ALP (FIG. 7F) of indicated mice. n=5-6. FIG. 7G. H&E staining liver sections of indicated mice as indicated therein. Scale bar=100 μm. Data are shown as mean±SEM. *, P<0.05, Student's unpaired t test.

FIGS. 8A-8U. AMPK deficiency exacerbates liver damage in AMLN-induced NASH. FIG. 8A Immunoblot (IB) analysis of liver lysate from ND and AMLN-fed C57BL/6J mice. n=3. FIGS. 8B-8E. Flox and ALKO mice were fed with AMLN diet for 30 weeks. FIGS. 8B-8D. Body weight (FIG. 8B), liver weight (FIG. 8C) and liver/body weight ratio (FIG. 8D) of indicated mice. n=9. FIG. 8E. Measurement of liver triglyceride (TG) of indicated mice on AMLN diet. n=9. FIG. 8F. Fasting blood glucose of indicated mice on AMLN diet for 10 weeks. n=6-7. FIG. 8G. Glucose (1.2 g/kg BW) tolerance test on indicated mice on AMLN diet for 10 weeks. n=6-7. (H) Insulin (1.2 U/kg BW) tolerance test on indicated mice on AMLN diet for 12 weeks. n=6-7. FIGS. 8I-8U. Flox and ALKO mice were fed with AMLN diet for 30 weeks. FIGS. 8I-8L. Expression of Adgre1, G6pc, Pck1, Srebf1, Fasn, Ppargc1a and Cpt1a in the liver of indicated mice on AMLN diet. n=9. FIGS. 8M-8O. Serum ALT (FIG. 8M), AST (FIG. 8N) and ALP (FIG. 8O) of indicated mice on AMLN diet. n=9. FIG. 8P. Paraffin-embedded liver sections of indicated mice were stained with TUNEL and DAPI as indicated therein; in the merged image sections, the different stains correspond to the grayscale shades as indicated in the single stained images to the left. Scale bar=50 μm. (Q) Quantification of the numbers of TUNEL-positive nuclei per field in liver sections shown in (FIG. 8P). n=7. FIG. 8R. H&E and Sirius red staining of liver sections from indicated mice as indicated therein. Scale bar=100 μm. FIG. 8S. Quantification of liver fibrosis area (% of total area) in liver sections shown in (FIG. 8R). n=7. FIG. 8T. Measurement of liver hydroxyproline of indicated mice. n=9. FIG. 8U. Expression of Tgfb, Timp1, Col1a1, Col3a1, Acta2, Pdgfa, Pdgfb, Pdgfra and Ddr2 in the liver of indicated mice. n=9. Data are shown as mean±SEM. *, P<0.05.

FIGS. 9A-9D. AMPK knockout increases caspase-6 cleavage and activation in AMLN-fed mice. FIG. 9A. Flox and LAKO mice were fed AMLN diet for 30 weeks. Immunoblot (IB) analysis of liver lysate from indicated mice. n=3. FIG. 9B. Expression of Casp6 in the liver of C57BL/6J mice on chow diet (ND), 11 weeks of CD-HFD and 30 weeks of AMLN diet. n=7-8. FIG. 9C. Flox and LAKO mice were fed AMLN diet for 30 weeks. Paraffin-embedded liver sections of indicated mice were stained with aCasp6 and DAPI as indicated therein; in the merged image sections, the different stains correspond to the grayscale shades as indicated in the single stained images to the left. Scale bar=50 μm. FIG. 9D. Quantification of the intensity of aCasp6 staining per field in liver sections shown in FIG. 9C. n=7. Data are shown as mean±SEM. *, P<0.05, Student's unpaired t test.

FIGS. 10A-10E. Liver damage at different time points of CD-HFD feeding. C57BL/6J mice were fed with CD-HFD for 3, 6, 8 and 11 weeks. FIG. 10A. H&E staining (as indicated therein) of liver sections from mice on CD-HFD for indicated time. Scale bar=100 μm. FIG. 10B. Paraffin-embedded liver sections of mice on CD-HFD for indicated time were stained with TUNEL and DAPI as indicated therein; in the merged image sections, the different stains correspond to the grayscale shades as indicated in the single stained images to the left. Scale bar=50 μm. n=4-5. FIGS. 10C-10E. Serum ALT (FIG. 10C), AST (FIG. 10D) and ALP (FIG. 10E) of indicated mice. n=4-5. Data are shown as mean±SEM.

FIGS. 11A-11G. Caspase-6 knockdown attenuates hepatic fibrosis. Flox and LAKO mice were fed with CD-HPD for 6 weeks. After 3 weeks of CD-HPD feeding, mice were intravenously injected with 1.5 mg/kg BW Caspase-6 siRNA (KD) or scrambled RNA (Sc) twice per week for 3 weeks along with continuous CD-HPD feeding. FIG. 11A. Schematic diagram of experimental design. FIG. 11B. Expression of Casp6 in the liver of indicated mice. n=7-10. FIG. 11C-11D. Body weight (FIG. 11C) and liver weight (FIG. 11D) of indicated mice. n=7-10. FIG. 11E. H&E and Sirius red staining of liver sections from indicated mice as indicated therein. Scale bar=100 μm. FIG. 11F. Quantification of liver fibrosis area (% of total area) in liver sections shown in FIG. 11E. n=7. FIG. 11G. Measurement of liver hydroxyproline of indicated mice. N=7-9. Data are shown as mean±SEM. *, P<0.05, two-way ANOVA.

FIG. 12. Liver morphology in mouse NASH models. H&E staining (as indicated therein) of liver sections from NASH mouse models in FIG. 3A. Scale bar=100 μm.

FIGS. 13A-13I. A-769662 improves liver damage through AMPK activation in hepatocytes in NASH. C57BL/6J mice were fed with CD-HFD for 8 weeks. After 6 weeks of CD-HFD feeding, mice were intraperitoneally injected with 25 mg/kg A-769662 or vehicle daily for 2 weeks along with continuous CD-HFD feeding. FIG. 13A. Schematic diagram of experimental design. FIG. 13B-13C. Body weight (FIG. 13B) and liver weight (FIG. 13C) of indicated mice. n=7. FIG. 13D. Measurement of liver triglyceride (TG) of indicated mice. n=7. FIG. 13E. Quantification of the numbers of TUNEL-positive nuclei per field in liver sections shown in (FIG. 10B (W6), 10A). n=5-7 *, P<0.05, Student's unpaired t test. FIG. 13F-13H. Flox and LAKO mice were fed with CD-HPD for 8 weeks. After 6 weeks of CD-HFD feeding, mice were intraperitoneally injected with 25 mg/kg A-769662 or vehicle daily for 2 weeks along with continuous CD-HFD feeding. FIG. 13F. Paraffin-embedded liver sections of indicated mice were stained with TUNEL and DAPI as indicated therein; in the merged image sections, the different stains correspond to the grayscale shades as indicated in the single stained images to the left. Scale bar=50 μm. FIG. 13G. Quantification of the numbers of TUNEL-positive nuclei per field in liver sections shown in FIG. 13F. FIG. 13H. Serum ALT of indicated mice. n=4-5. *, P<0.05, two-way ANOVA. FIG. 13I. Expression of Adgre1 and Ccl2 in the liver of indicated mice. n=7. Data are shown as mean±SEM. *, P<0.05, Student's unpaired t test.

FIGS. 14A-14G. Caspase-6 inhibitor attenuates hepatic fibrosis without effect on steatosis. Flox and LAKO mice were fed with CD-HFD for 8 weeks. After 6 weeks of CD-HPD feeding, mice were intraperitoneally injected with 5 mg/kg Z-VEID-FMK (VEID) (SEQ ID NO: 71) or vehicle every other day for 2 weeks along with continuous CD-HFD feeding. FIG. 14A. Schematic diagram of experimental design. FIGS. 14B-14C. Body weight (FIG. 14B) and liver weight (FIG. 14C) of indicated mice. n=5-6. FIG. 14D. Quantification of the numbers of TUNEL-positive nuclei per field in liver sections shown in (FIG. 10B (W6), 10J). n=5-6. FIG. 14E. H&E and Sirius red staining (as indicated therein) of liver sections from indicated mice. Scale bar=100 μm. FIG. 14F. Quantification of liver fibrosis area (% of total area) in liver sections shown in FIG. 14E. n=5-6. FIG. 14G. Measurement of liver hydroxyproline of indicated mice. n=5-6. Data are shown as mean±SEM. *, P<0.05, two-way ANOVA. FIGS. 14A-14G. disclose “VEID” as SEQ ID NO: 71.

FIGS. 15A-15F. AMPK phosphorylates caspase-6 and inhibits its cleavage. FIG. 15A. Caspase-6 activity was measure in the lysates from isolated primary hepatocytes treated with vehicle or 250 μM BSA-conjugated palmitic acid (PA) or 50 ng/ml TNFα for 2 hrs. Data are shown as mean±SD. *, P<0.05, Student's unpaired t test. FIG. 15B. HepG2 cells were pretreated with 40 μM A-769662 for 1 hr, and then treated with 30 μg/ml cycloheximide (CHX) and 50 ng/ml TNFα for 2 hrs. Cell lysates were subject to immunoblot (IB) analysis. FIG. 15C. In vitro kinase assay using purified caspase-6-mycexpressed in HEK293T cells, and recombinant AMPKα1β1γ1 active kinase. FIGS. 15D-15E. Flox and LAKO mice were fed with CD-HFD for 8 weeks. After 6 weeks of CD-HFD feeding, mice were intraperitoneally injected with 25 mg/kg A-769662 or vehicle daily for 2 weeks along with continuous CD-HFD feeding. FIG. 15D. IB analysis of liver lysate from indicated mice. S.E., shorter exposure; L.E., longer exposure. FIG. 15E. Quantification of IB analysis in FIG. 15D. n=3. *, P<0.05, two-way ANOVA. FIG. 15F. Quantification of IB analysis in FIG. 5K. n=3. Data are shown as mean±SEM. *, P<0.05, Student's unpaired t test.

FIGS. 16A-16D. Caspase-6 sustain activation of caspase cascade to induce apoptosis. FIG. 16A. HepG2 cells transfected with scrambled RNA or caspase-6 siRNA were treated with vehicle or 300 μg/ml CHX and 50 ng/ml TNFα for 20 hrs. Viability was measured by CCK-8 assay. Data are shown as mean±SD. *, P<0.05, two-way ANOVA. FIG. 16B. HepG2 cells transfected with scrambled RNA or Caspase-6 siRNA were treated with vehicle or 30 μg/ml CHX and 50 ng/ml TNFα for 2 hrs. Medium was changed to remove treatment. Cells were harvested at indicated time points. Cell lysates were subject to IB analysis. FIG. 16C. Isolated primary hepatocytes were pretreated with 25 μM Z-VEID-FMK (VEID) (SEQ ID NO: 71) for 1 hr and treated with 30 μg/ml CHX and 50 ng/ml TNFα for 2 hrs. Medium was changed to remove treatment for 4 hrs. Cell lysates were subject to IB analysis. FIG. 16D. Proposed model for roles of AMPK/caspase-6 axis in the regulation of NASH-associated liver damage.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAINED DESCRIPTION OF THE INVENTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Aspects of the present disclosure will employ, unless otherwise indicated, techniques of molecular biology, microbiology, organic chemistry, biochemistry, physiology, cell biology, blood vessel biology, and the like, which are within the skill of the art. Such techniques are explained fully in the literature.

Prior to describing the various aspects of the present disclosure, the following definitions are provided and should be used unless otherwise indicated. Additional terms may be defined elsewhere in the present disclosure.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

As used in the specification and the appended claims, the singular forms “a,” “an” and “the” include plural referents unless the context clearly dictates otherwise. Thus, for example, reference to “a therapeutic agent,” “a therapeutic agent that targets the AMPK/caspase-6 axis,” or “a caspase-6 inhibitor,” including, but not limited to, two or more such therapeutic agents, therapeutic agents that target the AMPK/caspase-6 axis, or caspase-6 inhibitors, including combinations of therapeutic agents, therapeutic agents that target the AMPK/caspase-6 axis, or caspase-6 inhibitors, and the like.

Reference to “a/an” chemical compound, therapeutic agent, and pharmaceutical composition each refers to one or more molecules of the chemical compound, therapeutic agent, and pharmaceutical composition rather than being limited to a chemical compound, therapeutic agent, and pharmaceutical composition, the one or more molecules may or may not be identical, so long as they fall under the category of the chemical compound, therapeutic agent, and pharmaceutical composition. Thus, for example, “a” therapeutic agent is interpreted to include one or more molecules of the therapeutic agent, where the therapeutic agent molecules may or may not be identical (e.g., comprising different isotope abundances and/or different degrees of hydration or in equilibrium with different conjugate base or conjugate acid forms).

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

As used interchangeably herein, “subject,” “individual,” or “patient” can refer to a vertebrate organism, such as a mammal (e.g. human) “Subject” can also refer to a cell, a population of cells, a tissue, an organ, or an organism, preferably to human and constituents thereof.

As used herein, “liver diseases” are acute or chronic damages to the liver based in the duration of the disease. The liver damage may be caused by infection, injury, exposure to drugs or toxic compounds such as alcohol or impurities in foods, an abnormal build-up of normal substances in the blood, an autoimmune process, a genetic defect (such as haemochromatosis), or other unknown causes. Exemplary liver diseases include, but are not limited to, cirrhosis, liver fibrosis, non-alcoholic fatty liver disease (NAFLD), non-alcoholic steatohepatitis (NASH), alcoholic steatohepatitis (ASH), hepatic ischemia reperfusion injury, primary biliary cirrhosis (PBC), and hepatitis, including both viral and alcoholic hepatitis.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a liver disease associated with the AMPK/caspase-6 axis. The effect can be therapeutic in terms of a partial or complete cure of a disease, condition, symptom or adverse effect attributed to the disease, disorder, or condition. The term “treatment” as used herein can include any treatment of liver disease associated with the AMPK/caspase-6 axis in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

It is understood, that unless otherwise specified, temperatures referred to herein are based on atmospheric pressure (i.e. one atmosphere).

### Methods of Treating a Liver Disease by Target the AMPK/Caspase-6 Axis

The present disclosure provides that caspase-6 serves a crucial role in steatosis-induced hepatic cell death and integrates signals from both inflammation and changes in energy status via direct phosphorylation by AMPK. The present disclosure further provides that once AMPK activity declines, caspase-6 becomes activated, and in turn cleaves Bid to induce sustained cytochrome c release in a feedforward loop that leads to hepatocyte death. This direct link to obesity suggests that the AMPK/caspase-6 axis plays a key role in the development of NASH and represents a new site for therapeutic intervention. As used herein, “a therapeutic agent that targets AMPK/Caspase-6 axis” refers to a therapeutic agent, e.g., a chemical compound, an antibody, a DNA molecule, an RNAi molecule, or other pharmaceutically active agent that modulates at least one protein in the AMPK/Caspase-6 axis, e.g., an inhibits Caspase-6 or an agonist of AMPK.

Accordingly, disclosed herein is a method of treating and/or preventing liver disease in a patient in need thereof, by targeting the AMPK/caspase-6 axis to inhibit caspase-6 activity and/or to activate AMPK activity. The liver disease can be any liver disease, including, but not limited to, chronic and/or metabolic liver diseases, nonalcoholic fatty liver disease (NAFLD), and nonalcoholic steatohepatitis (NASH).

In certain embodiments, provided herein is a method of treating and/or preventing nonalcoholic steatohepatitis (NASH) in a patient in need thereof, comprising administering to the patient a therapeutically effective amount of an therapeutic agent that targets the AMPK/caspase-6 axis. In certain embodiments, the therapeutic agent is an inhibitor that inactivates and/or decrease caspase-6 activity. In other embodiments, the therapeutic agent is an activator that activates and/or increases AMPK activity. The present disclosure encompasses any inhibitors and/or activators, now known and/or later developed, that are capable of targeting the AMPK/caspase-6 axis to inhibit caspase-6 activity and/or to activate AMPK activity.

In the methods provided herein, the therapeutic agent that targets the AMPK/caspase-6 axis can be a single and/or a combination of at least one such inhibitor and/or activator. In certain embodiments, the therapeutic agent of the present disclosure can be administered in a single pharmaceutical composition, or separately in more than one pharmaceutical composition. Accordingly, also provided herein is a pharmaceutical composition comprising a therapeutically effective amount of single and/or a combination of at least one therapeutic agent that targets the AMPK/caspase-6 axis.

Non-alcoholic fatty liver disease (NAFLD) is the buildup of extra fat in liver cells that is not caused by alcohol. NAFLD may cause the liver to swell (i.e. steatohepatitis), which in turn may cause scarring (i.e. cirrhosis) over time and may lead to liver cancer or liver failure. NAFLD is characterized by the accumulation of fat in hepatocyes and is often associated with some aspects of metabolic syndrome (e.g. type 2 diabetes mellitus, insulin resistance, hyperlipidemia, and hypertension). The frequency of this disease has become increasingly common due to consumption of carbohydrate-rich and high fat diets. A subset (about 20%) of NAFLD patients develop nonalcoholic steatohepatitis (NASH).

NASH, a subtype of fatty liver disease, is the more severe form of NAFLD. It is characterized by macrovesicular steatosis, balloon degeneration of hepatocytes, and/or inflammation ultimately leading to hepatic scarring (i.e. fibrosis). Patients diagnosed with NASH progress to advanced stage liver fibrosis and eventually cirrhosis. The current treatment for cirrhotic NASH patients with end-stage disease is liver transplant.

A study has shown that a significant proportion of diagnosed NASH patients (39%) have not had a liver biopsy to confirm the diagnosis. A greater proportion of diagnosed NASH patients have metabolic syndrome parameters than what is reported in the literature (type-II diabetes mellitus 54%, Obesity 71%, metabolic syndrome 59%). 82% of physicians use a lower threshold value to define significant alcohol consumption compared with practice guideline recommendations. 88% of physicians prescribe some form of pharmacologic treatment for NASH (Vit E: prescribed to 53% of NASH patients, statins: 57%, metformin: 50%). Therefore, the vast majority of patients are prescribed medications despite a lack of a confirmed diagnosis or significant data to support the intervention and alcohol thresholds to exclude NASH are lower than expected.

Another common liver disease is primary sclerosing cholangitis (PSC). It is a chronic or long-term liver disease that slowly damages the bile ducts inside and outside the liver. In patients with PSC, bile accumulates in the liver due to blocked bile ducts, where it gradually damages liver cells and causes cirrhosis, or scarring of the liver. Currently, there is no effective treatment to cure PSC. Many patients having PSC ultimately need a liver transplant due to liver failure, typically about 10 years after being diagnosed with the disease. PSC may also lead to bile duct cancer.

Liver fibrosis is the excessive accumulation of extracellular matrix proteins, including collagen that occurs in most types of chronic liver diseases. Advanced liver fibrosis results in cirrhosis, liver failure, and portal hypertension and often requires liver transplantation.

Treatment and/or Prevention Method

Disclosed herein is a method of treating and/or preventing liver disease in a patient in need thereof, comprising administering to the patient a therapeutically effective amount of an inhibitor that targets the AMPK/caspase-6 axis by inhibiting caspase-6 via activating AMPK. The presence of active liver disease can be detected by the existence of elevated enzyme levels in the blood. Specifically, blood levels of alanine aminotransferase (ALT) and aspartate aminotransferase (AST), above clinically accepted normal ranges, are known to be indicative of on-going liver damage. Routine monitoring of liver disease patients for blood levels of ALT and AST is used clinically to measure progress of the liver disease while on medical treatment. Reduction of elevated ALT and AST to within the accepted normal range is taken as clinical evidence reflecting a reduction in the severity of the patients' on-going liver damage.

In certain embodiments, the liver disease is a chronic liver disease. Chronic liver diseases involve the progressive destruction and regeneration of the liver parenchyma, leading to fibrosis and cirrhosis. In general, chronic liver diseases can be caused by viruses (such as hepatitis B, hepatitis C, cytomegalovirus (CMV), or Epstein Barr Virus (EBV)), toxic agents or drugs (such as alcohol, methotrexate, or nitrofurantoin), a metabolic disease (such as non-alcoholic fatty liver disease (NAFLD), non-alcoholic steatohepatitis (NASH), haemochromatosis, or Wilson's Disease), an autoimmune disease (such as Autoimmune Chronic Hepatitis, Primary Biliary Cirrhosis, or Primary Sclerosing Cholangitis), or other causes (such as right heart failure).

In one embodiment, provided herein is a method for reducing the level of cirrhosis. In one embodiment, cirrhosis is characterized pathologically by loss of the normal microscopic lobular architecture, with fibrosis and nodular regeneration. Methods for measuring the extent of cirrhosis are well known in the art. In one embodiment, the level of cirrhosis is reduced by about 5% to about 100%. In one embodiment, the level of cirrhosis is reduced by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, at least about 95%, or about 100% in the subject.

In certain embodiments, the liver disease is a metabolic liver disease. In one embodiment, the liver disease is non-alcoholic fatty liver disease (NAFLD). NAFLD is associated with insulin resistance and metabolic syndrome (obesity, combined hyperlipidemia, diabetes mellitus (type II) and high blood pressure). NAFLD is considered to cover a spectrum of disease activity and begins as fatty accumulation in the liver (hepatic steatosis).

It has been shown that both obesity and insulin resistance probably play a strong role in the disease process of NAFLD. In addition to a poor diet, NAFLD has several other known causes. For example, NAFLD can be caused by certain medications, such as amiodarone, antiviral drugs (e.g., nucleoside analogues), aspirin (rarely as part of Reye's syndrome in children), corticosteroids, methotrexate, tamoxifen, or tetracycline. NAFLD has also been linked to the consumption of soft drinks through the presence of high fructose corn syrup which may cause increased deposition of fat in the abdomen, although the consumption of sucrose shows a similar effect (likely due to its breakdown into fructose). Genetics has also been known to play a role, as two genetic mutations for this susceptibility have been identified.

If left untreated, NAFLD can develop into non-alcoholic steatohepatitis (NASH), which is the most extreme form of NAFLD, a state in which steatosis is combined with inflammation and fibrosis. NASH is regarded as a major cause of cirrhosis of the liver of unknown cause. Accordingly, provided herein is a method of treating and/or preventing nonalcoholic steatohepatitis (NASH) in a patient in need thereof, comprising administering to the patient a therapeutically effective amount of an inhibitor that targets the AMPK/caspase-6 axis by inhibiting caspase-6 via activating AMPK.

Also provided herein is a method of treating and/or preventing liver fibrosis in a patient in need thereof, comprising administering to the patient a therapeutically effective amount of an inhibitor that targets the AMPK/caspase-6 axis by inhibiting caspase-6 via activating AMPK. Liver fibrosis is the excessive accumulation of extracellular matrix proteins including collagen that occurs in most types of chronic liver diseases. In certain embodiments, advanced liver fibrosis results in cirrhosis and liver failure. Methods for measuring liver histologies, such as changes in the extent of fibrosis, lobular hepatitis, and periportal bridging necrosis, are well known in the art.

In one embodiment, the level of liver fibrosis, which is the formation of fibrous tissue, fibroid or fibrous degeneration, is reduced by more than about 90%. In one embodiment, the level of fibrosis, which is the formation of fibrous tissue, fibroid or fibrous degeneration, is reduced by at least about 90%, at least about 80%, at least about 70%, at least about 60%, at least about 50%, at least about 40%, at least about 30%, at least about 20%, at least about 10%, at least about 5% or at least about 2%.

In one embodiment, the inhibitor provided herein reduces the level of fibrogenesis in the liver. Liver fibrogenesis is the process leading to the deposition of an excess of extracellular matrix components in the liver known as fibrosis. It is observed in a number of conditions such as chronic viral hepatitis B and C, alcoholic liver disease, drug-induced liver disease, hemochromatosis, auto-immune hepatitis, Wilson disease, primary biliary cirrhosis, sclerosing cholangitis, liver schistosomiasis and others. In one embodiment, the level of fibrogenesis is reduced by more than about 90%. In one embodiment, the level of fibrogenesis is reduced by at least about 90%, at least about 80%, at least about 70%, at least about 60%, at least about 50%, at least 40%, at least about 30%, at least about 20%, at least about 10%, at least about 5% or at least 2%.

In still other embodiments, provided herein is a method of treating and/or preventing primary sclerosing cholangitis (PSC) in a patient in need thereof, comprising administering to the patient a therapeutically effective amount of an inhibitor that targets the AMPK/caspase-6 axis by inhibiting caspase-6 via activating AMPK.

### Agent/Compound Targeting the AMPK/Caspase-6 Axis

As disclosed herein, activation of AMPK or inhibition of caspase-6, even after the onset of NASH, improves liver damage and fibrosis. Therefore, in some embodiments, the therapeutic agent for use in the disclosed compositions and methods is a caspase-6 inhibitor. This inhibitor can be a pan caspase inhibitor, such as Z-VAD-FMK. In some embodiments, the therapeutic agent of the disclosed compositions and methods is a selective caspase-6 inhibitor, such as Z-VEID-FMK (“VEID” disclosed as SEQ ID NO: 71). In addition, U.S. Pat. No. 8,324,173 is incorporated by reference for peptides useful dual caspase-2/-6 inhibitors. U.S. Pat. No. 8,518,942 is incorporated by reference for caspase-3/-6 inhibiting molecules. Caspase-6 can also be targeted using oligonucleotides, such as described in U.S. Pat. No. 6,566,135, which is incorporated by reference for the teaching of caspose-6 antisense compounds.

In some embodiments, the therapeutic agent of the disclosed methods is an AMPK activator, such as A-769662. Activation of AMPK may be induced by indirect activators such as Metformin, Thiazolidinediones such as troglitazone, rosiglitazone or pioglitazone, Adiponectin, Leptin, Ciliary Neurotrophic Factor (CNTF), Ghrelin/cCannabinoids, Interleukin-6, natural products such as alpha-Lipoic Acid alkaloids, bitter melon extracts, resveratrol, epigallocathechin gallate, berberine, quercetin, ginsenoside, curcumin, caffeic acid phenethyl ester, theaflavin. Activation of AMPK may be induced by direct Activators such as A-769662 (Cool, B., et al. (2006). Cell Metab. 3, 403-416) or PT1 (Pang et al. (2008) J. Biol. Chem. 283, 16051-16060). Examples of thienopyridone derivatives that can be used as AMPK activators are disclosed in WO2009135580, WO2009124636, US20080221088, and EP1754483, which are incorporated by reference for these derivatives. Examples of imidazole derivatives are disclosed in WO2008120797 and EP2040702 which discloses, which are incorporated by reference for these derivatives. Examples of thiazole derivatives are disclosed in EP1907369 which discloses, which are incorporated by reference for these derivatives. In some embodiments, the AMPK activator is metformin or a thiazolidinedione, such as for example troglitazone, rosiglitazone or pioglitazone.

### Dosing and Administration

While it is possible for an active ingredient to be administered alone, it may be preferable to present them as pharmaceutical formulations or pharmaceutical compositions as described below. The formulations, both for veterinary and for human use, of the disclosure comprise at least one of the active ingredients, together with one or more acceptable carriers therefor and optionally other therapeutic ingredients. The carriers must be “acceptable” in the sense of being compatible with the other ingredients of the formulation and physiologically innocuous to the recipient thereof.

Each of the active ingredients can be formulated with conventional carriers and excipients, which will be selected in accord with ordinary practice. Tablets can contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10. The therapeutically effective amount of active ingredient can be readily determined by a skilled clinician using conventional dose escalation studies. Typically, the active ingredient will be administered in a dose from 0.01 milligrams to 2 grams. In one embodiment, the dosage will be from about 10 milligrams to 450 milligrams. In another embodiment, the dosage will be from about 25 to about 250 milligrams. In another embodiment, the dosage will be about 50 or 100 milligrams. In one embodiment, the dosage will be about 100 milligrams. It is contemplated that the active ingredient may be administered once, twice or three times a day. Also, the active ingredient may be administered once or twice a week, once every two weeks, once every three weeks, once every four weeks, once every five weeks, or once every six weeks.

The pharmaceutical composition for the active ingredient can include those suitable for the foregoing administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste. In certain embodiments, the active ingredient may be administered as a subcutaneous injection.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, or surface active agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

The active ingredient can be administered by any route appropriate to the condition. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. In certain embodiments, the active ingredients are orally bioavailable and can therefore be dosed orally. In one embodiment, the patient is human.

### Pharmaceutical Compositions

The pharmaceutical compositions of the disclosure provide for an effective amount of an inhibitor that targets the AMPK/caspase-6 axis by inhibiting caspase-6 by activating AMPK.

When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as, for example, calcium or sodium carbonate, lactose, lactose monohydrate, croscarmellose sodium, povidone, calcium or sodium phosphate; granulating and disintegrating agents, such as, for example, maize starch, or alginic acid; binding agents, such as, for example, cellulose, microcrystalline cellulose, starch, gelatin or acacia; and lubricating agents, such as, for example, magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as, for example, glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as, for example, peanut oil, liquid paraffin or olive oil.

Aqueous suspensions of the disclosure contain the active materials in admixture with excipients suitable for the manufacture of aqueous suspensions. Such excipients include a suspending agent, such as, for example, sodium carboxymethylcellulose, methylcellulose, hydroxypropyl methylcelluose, sodium alginate, polyvinylpyrrolidone, gum tragacanth and gum acacia, and dispersing or wetting agents such as, for example, a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as, for example, ethyl or n-propyl p-hydroxy-benzoate, one or more coloring agents, one or more flavoring agents and one or more sweetening agents, such as, for example, sucrose or saccharin.

Oil suspensions may be formulated by suspending the active ingredient in a vegetable oil, such as, for example, arachis oil, olive oil, sesame oil or coconut oil, or in a mineral oil such as, for example, liquid paraffin. The oral suspensions may contain a thickening agent, such as, for example, beeswax, hard paraffin or cetyl alcohol. Sweetening agents, such as, for example, those set forth above, and flavoring agents may be added to provide a palatable oral preparation. These compositions may be preserved by the addition of an antioxidant such as, for example, ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as, for example, olive oil or arachis oil, a mineral oil, such as, for example, liquid paraffin, or a mixture of these. Suitable emulsifying agents include naturally-occurring gums, such as, for example, gum acacia and gum tragacanth, naturally occurring phosphatides, such as, for example, soybean lecithin, esters or partial esters derived from fatty acids and hexitol anhydrides, such as, for example, sorbitan monooleate, and condensation products of these partial esters with ethylene oxide, such as, for example, polyoxyethylene sorbitan monooleate. The emulsion may also contain sweetening and flavoring agents. Syrups and elixirs may be formulated with sweetening agents, such as, for example, glycerol, sorbitol or sucrose. Such formulations may also contain a demulcent, a preservative, a flavoring or a coloring agent.

The pharmaceutical compositions of the disclosure may be in the form of a sterile injectable preparation, such as, for example, a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as, for example, a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as, for example, oleic acid may likewise be used in the preparation of injectables.

The amount of active ingredient that may be combined with the carrier material to produce a single dosage form will vary depending upon the host treated and the particular mode of administration, such as oral administration or subcutaneous injection. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 □g of the active ingredient per milliliter of solution in order that infusion of a suitable volume at a rate of about 30 mL/hr can occur. When formulated for subcutaneous administration, the formulation is typically administered about twice a month over a period of from about two to about four months.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations can be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In certain embodiments, the inhibitor of the present disclosure may be formulated in any suitable dosage form for an appropriate administration. In certain embodiments, the methods provided herein comprise administering a pharmaceutical composition comprising the inhibitor of the present disclosure and a pharmaceutically acceptable carrier or excipient. Combination formulations and/or treatment according to the present disclosure comprise the inhibitor of the present disclosure together with one or more pharmaceutically acceptable carriers or excipients and optionally other therapeutic agents, now known or later developed, for treating and/or preventing a liver disease, particularity NASH. Combination formulations containing the active ingredient may be in any form suitable for the intended method of administration.

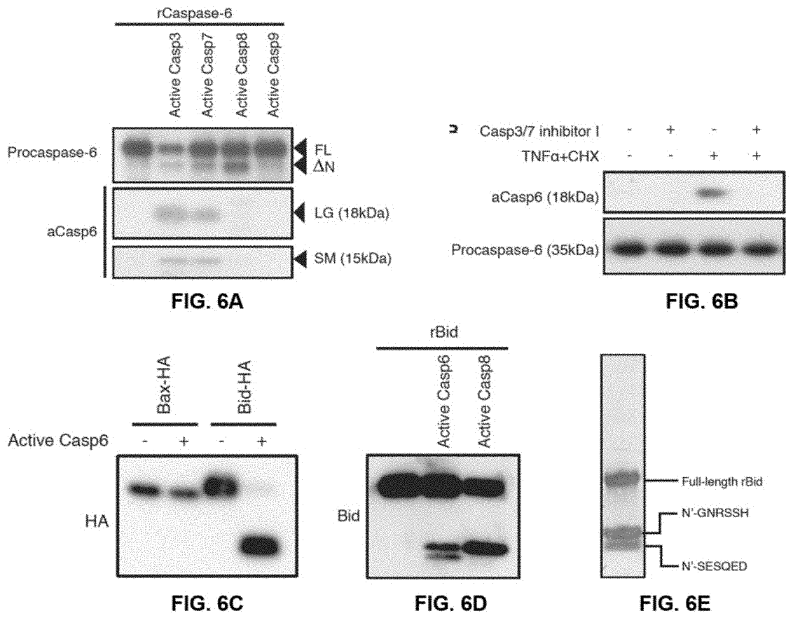

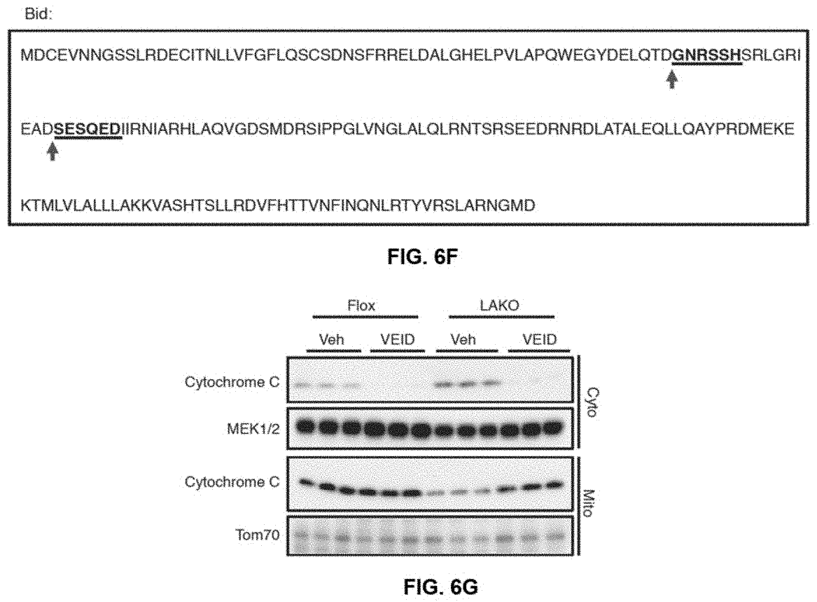

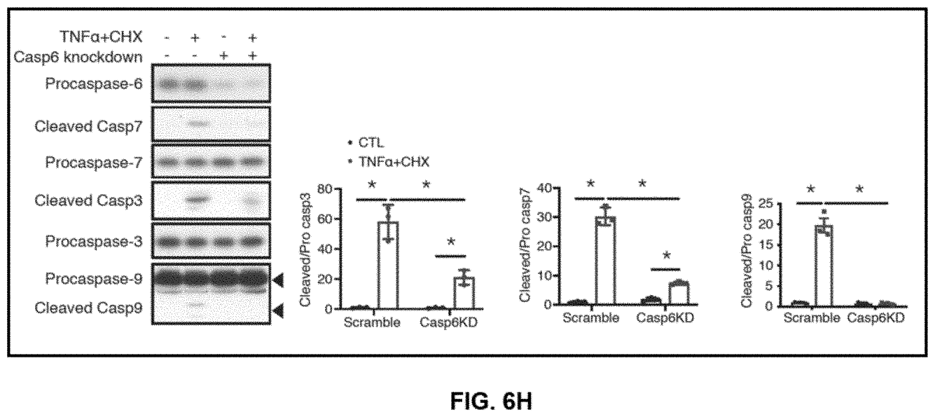

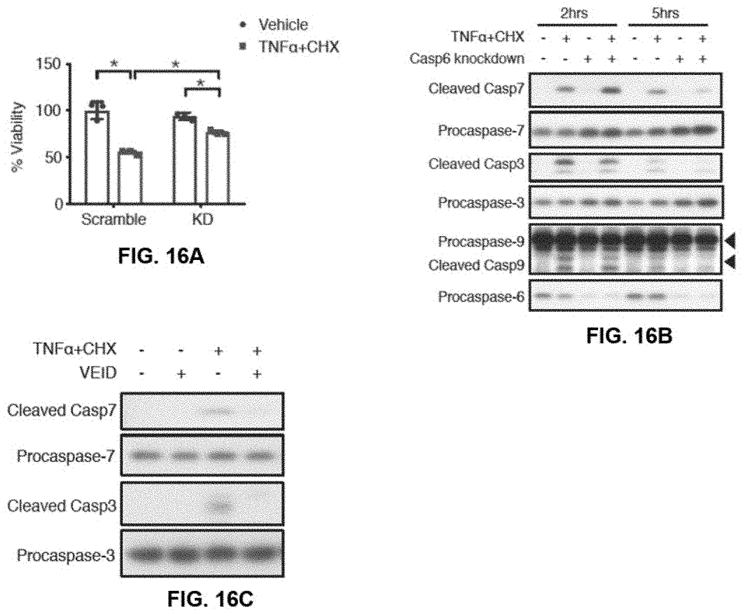

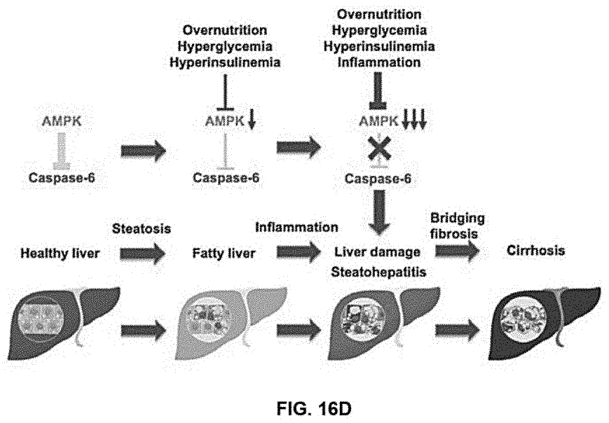

Overnutrition-induced hepatic steatosis and inflammation lead to liver damage in NASH(2). The present disclosure provides that an AMPK-caspase-6 axis that regulates hepatocellular apoptosis and may shed new light on the “two-hit” or “multiple-hit” of NASH. Inflammation in NAFLD leads to caspase-6 activation by the increased activity of upstream executioners, caspase-3 and -7. Active caspase-6 in turn cleaves Bid to increase mitochondrial cytochrome c release in a feedforward loop, in which activation of upstream caspases is persistent, such that the apoptotic caspase cascade is sustained in hepatocytes. However, in the metabolically healthy liver, AMPK activity is maintained to allow phosphorylation of procaspase-6, which inhibits its activation, thus preventing this feedforward loop (FIG. 6I, FIG. 16D). When AMPK activity is reduced by overnutrition, hyperglycemia, hyperinsulinemia and inflammation in obesity, diabetes and NAFL, caspase-6 is de-repressed, leading to activation of the feedforward loop, priming hepatocytes for caspase-mediated apoptosis (FIG. 16D) (28). AMPK inhibition thus serves as a point of convergence by which overnutrition, steatosis, hyperinsulinemia and inflammation contribute to liver damage. If so, pharmaceutical interventions that specifically activate AMPK or block caspase-6 in livers could represent new approaches to treat NASH.

Caspase-2 triggers de novo lipogenesis and steatosis during NAFL (19). In contrast, caspase-6 does not contribute to the development of steatosis, but specifically mediates NASH-associated liver damage. Although knockout of caspase-3 and -8 also protects against hepatocyte apoptosis (29, 30), global knockout of caspase-8 is embryonically lethal, whereas caspase-3 whole body knockout leads to multiple developmental defects (31, 32). In contrast, caspase-6 deficient mice exhibit no developmental defects (14). It is possible that specifically targeting caspase-6 could be an effective therapeutic strategy with fewer side effects.

In certain embodiments, the present disclosure provides that, in AMLN and CD-HFD fed mice, both of which exhibit characteristics of human NASH, LAKO exaggerates liver damage without affecting steatosis and inflammation. Exacerbation of liver damage leads increased scarring and fibrosis. Two-weeks treatment with both AMPK activator and caspase-6 inhibitor substantially reduced hepatocellular death and hepatic fibrosis. Activation of AMPK inhibits proliferation of hepatic stellate cells (HSCs) (33). Thus, the effects of the AMPK activator on hepatic fibrosis could be attributed to both reduction of liver damage and inhibition of HSCs.

In other embodiments of the present disclosure, caspase-6 activity with the VEID-pNA (“VEID” disclosed as SEQ ID NO: 71) substrate was measured, and Z-VEID-FMK (“VEID” disclosed as SEQ ID NO: 71) was used as a caspase-6 inhibitor. While VEID (SEQ ID NO: 71) is a preferred substrate of caspase-6, it cross-reacts to a lesser extent with caspase-3 (34, 35). To ensure specificity, multiple methodologies were utilized to determine activation of caspase-6, including western blot and immunofluorescent staining of aCasp6. siRNA was also used to specifically deplete caspase-6, resulting in attenuation of liver damage in NASH. Taken together, the data presented in the present disclosure provide that caspase-6 is activated and participates in the pathogenesis of liver damage in NASH. Moreover, depletion of caspase-6 abrogated the exaggerated liver damage in LAKO mice, indicating that the AMPK-caspase-6 axis regulates liver damage.

Previous study showed that inhibition of caspase-3 and -7 attenuates Bid cleavage, suggesting the existence of a feedforward loop acting downstream of the executioner caspases (36). The present disclosure provides an elaborated role of caspase-6 in sustaining activation of the caspase cascade in this feedforward loop. Previous study demonstrated that caspase-6 cleaves Lamin A to induce nuclear/chromatin condensation in apoptosis (37). Although some nuclear localization of active caspase-6 in human and murine NASH samples were observed, most of active caspase-6 appeared to locate within the cytoplasm, suggesting that cleavage of Bid could be a dominant function of caspase-6. However, since caspase-6 does not participate in the initiating activation of the caspase cascade, it may play a role in mediating apoptosis only in chronic diseases. Caspase-6 has been proposed as an important target in Alzheimer' s disease (38, 39), which is also characterized by reduced AMPK activity (40). Thus, the AMPK-caspase-6 axis might have a role in other chronic inflammatory pathogenic processes.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Materials and Methods

### Mice

Prkaa1^{fl/fl} and Prkaa2^{fl/fl} mice were bred with albumin-cre mice to generate hepatocyte-specific AMPKα1/α2 double knockout (LAKO) mice in the C57BL/6J background. During the study, ear tag numbers were used to identify animals. Flox and knockout mice are littermates and cage mates. Researchers performing test and collecting data were blinded during experiments. Animals in each cohort were produced from 20 breeding pairs to minimize the birthdate range. Mice were housed in a specific pathogen-free facility with a 12-h light, 12-h dark cycle, and given free access to food and water, except for fasting period. Mice were used in accordance with the Guide for Care and Use of Laboratory Animals of the National Institute of Health. The protocols were approved by the Institutional Animal Care and Use Committee of (IACUC) of UCSD. Only male mice were used in the study.

Mice were fed with AMLN diet (Research Diet, Cat. D09100301) consisting of 40% Fat, 20% Fructose and 2% cholesterol for 30 weeks, or CD-HPD (Research Diet, Cat. A06071302) consisting of 60% Fat, 0.1% Methionine and no added Choline for 3 to 11 weeks. In the STAM model, C57BL/6J mice were injected with 200 μg streptozotocin (STZ) 2 days after birth and fed with 60% high-fat diet for 6 weeks, starting at 4 weeks of age. MUP-uPA mice were fed with 60% high-fat diet for 16 weeks starting at 8 weeks of age. In the Casp6 knockdown model, both Flox and LAKO mice were fed with CD-HFD for three weeks, and intravenously injected with scrambled (Ambion In Vivo, Cat. 4457289) or Casp6 siRNA (Ambion In Vivo, Cat. 4457310) with Invivofectamine 3.0 (Life Technologies, Cat. IVF3005) twice per week for three weeks, according to manufacturer's instruction.

### Primary Hepatocyte Isolation

Primary hepatocytes were isolated from 6-week old male mice by a 2-step collagenase perfusion method. Liver was perfused with HBSS (Life Technologies), and then with HBSS digestion buffer containing 0.3 mg/mL collagenase I and 2 tablet/100 mL protease inhibitor. After perfusion, cells were smashed through 100 μm strainer and washed with Williams' Medium E (Gibco). Hepatocytes were isolated by density gradient centrifugation using Percoll (Sigma Aldrich). Hepatocytes with 95% viability were cultured in Williams' Medium E with 5% serum and 15 mM HEPES at 37° C.

### Human Liver Sections

All human samples were collected under protocols approved by the UCSD Human Research Protections Program. Patient biopsy sections used in the study were obtained at UCSD. Kleiner Fibrosis score was given by an experienced liver pathologist in a double blinded manner Normal liver section slides were purchased from Genetex (Cat. GTX24348). Cirrhotic liver section slides were purchased from Novus Biologicals (Cat. NBP2-30273).

### In Vivo Administration of AMPK Agonist

AMPK agonist A-769662 (Cayman, Cat. 11900) was dissolved with solvent containing 1% DMSO, 30% polyethylene glycerol and 1% Tween-80. Mice were fed with CD-HFD for six weeks, then intraperitoneally injected with vehicle or 25 mg/kg A-769662 daily for two weeks.

### In Vivo Administration of Caspase-6 Inhibitor

Flox and LAKO mice were fed with CD-HFD for six weeks, then intraperitoneally injected with vehicle or 5 mg/kg Caspase-6 inhibitor Z-VEID-FMK (“VEID” disclosed as SEQ ID NO: 71) (R&D Systems, Cat. FMK006) every other day for two weeks.

### Glucose Tolerance Test

Fasting blood glucose were measured after 12 hrs fast, using Easy Step Blood Glucose Monitoring System. Mice were then intraperitoneally injected with D-[+]-glucose (Sigma) at a dose of 1.2 g/kg BW. Blood glucose levels were measured at 15, 30, 45, 60, 90 and 120 min after injection (11).

### Insulin Tolerance Test

Fasting blood glucose were measured after 4 hrs fast, using Easy Step Blood Glucose Monitoring System. Mice were then intraperitoneally injected with insulin (Humulin R) at a dose of 1.2 g/kg BW. Blood glucose levels were measured at 15, 30, 45, 60, 90 and 120 min after injection (11).

### TUNEL Staining

Paraffin-embedded liver sections were subject to de-paraffinization, and then stained with the ApoBrdU DNA fragmentation kit (Biovision, Cat. K401/K404), according to the manufacturer's instruction. Stained tissue was visualized with Zeiss LSM880 or Keyence Fluorescent Microscope.

### Active Caspase-6 Staining

Paraffin-embedded tissue sections were subjected to de-paraffinization and rehydration, and then were immersed in 95° C. antigen retrieval buffer (10 mM sodium citrate, 0.05% Tween 20, pH 6.0). Tissue sections were blocked with 1% normal donkey serum, and then stained with active caspase-6 antibody (Genetex, Cat. GTX59553) for 12 hrs at 4° C. Stained tissue was visualized with Zeiss LSM880 or Keyence Fluorescent Microscope.

### Fibrosis Staining

Paraffin-embedded liver sections were subject to de-paraffinization, and then stained with Sirius Red (Sigma Aldrich)/Fast Green (Fisher Scientific). Images were taken with NanoZoomer Slide Scanner.

### Caspase-6 Activity Measurement

Cell/tissue was homogenized with lysis buffer. Caspase-6 activity was determined with Caspase-6 Colorimetric Assay Kit (Biovision, Cat. K115), according to manufacturer's instruction. Caspase-6 activity was normalized to total protein amount.

### Triglyceride Measurement

Blood/Tissue triglyceride levels were determined using the Triglyceride Quantification Colorimetric/Fluorometric Kit (Biovision, Cat. K622) according to the manufacturer's instruction. Tissue triglyceride levels were normalized to tissue weight.

### ALT, AST, ALP Activity Measurement

Serum ALT activity was measured with Alanine Aminotransferase Activity Colorimetric/Fluorometric Assay Kit (Biovision, Cat. K752); AST activity was measured with Aspartate Aminotransferase Activity Colorimetric Assay Kit (Biovision, Cat. K753); ALP was measured with Alkaline Phosphatase Activity Colorimetric Assay Kit (Biovision, Cat. K412), according to the manufacturer's instruction.

### Hydroxyproline Measurement

Liver tissue was homogenized in distilled water. 100 μl lysate was hydrolyzed with 100 μl 10N NaOH at 120° C. for 1 hour. Lysate was cooled on ice and neutralized with 10N HCl. Hydroxyproline in supernatant was measured with Hydroxyproline assay kit (Abcam, Cat. Ab222941), according to the manufacturer's instruction.

### Western Blot

Western blot was performed as previously described(11). Briefly, tissues or cells were homogenized in a non-denaturing lysis buffer that contained 10 mM Tris (pH 7.4), 150 mM NaCl, 1 mM EGTA, 1 mM EDTA, 1% Triton X-100, 0.5% Nonidet P-40, with freshly added protease inhibitors tablet (Roche), and phosphatase inhibitor 2 mM sodium vanadate and 5 mM sodium fluoride. The extract was centrifuged at 9500 g for 10 mM at 4° C. Supernatants were collected and analyzed with BCA (Thermo Scientific) to quantify protein content. Proteins were resolved by SDS-PAGE and transferred to nitrocellulose membranes (Bio-Rad). Individual proteins were detected with specific antibodies and visualized on film using horseradish peroxidase-conjugated secondary antibodies (Fisher Scientific) and SuperSignal West Pico PLUS Chemilunminescent Substrate (Thermo Scientific).

### Gene Expression Analysis

Analysis of gene expression was performed as previously described (11). Tissues were homogenized in TRIzol Retherapeutic agent (Life Technologies). RNA was isolated with PureLink RNA mini kit (Life Technologies). 1 μg of purified RNA was used for reverse transcription-PCR to generate cDNA using High-Capacity cDNA Reverse Transcription Kit (Applied Biosystem). ΔΔCt real time PCR with Power SYBR Green PCR Master Mix (Life Technologies) and QuantStudio 5 Real-Time PCR System were used to analyze cDNA. Ppia (Cyclophilin A) was used as endogenous control.

Q-PCR primer sequences

| | | SEQ | | SEQ |
|---|---|---|---|---|
| | | ID | | ID |
| Gene | Forward Primer | NO: | Reverse Primer | NO: |
| Acta2 | GTCCCAGACATCAGGGAG | 1 | TCGGATACTTCAGCGTCAG | 2 |
| | TAA | | GA | |
| Adgre1 | CCCCAGTGTCCTTACAGAG | 3 | GTGCCCAGAGTGGATGTCT | 4 |
| | TG | | | |
| Casp3 | TGGTGATGAAGGGGTCATT | 5 | TTCGGCTTTCCAGTCAGAC | 6 |
| | TATG | | TC | |
| Casp6 | GGAAGTGTTCGATCCAGCC | 7 | GGAGGGTCAGGTGCCAAA | 8 |
| | G | | AG | |
| Casp8 | TGCTTGGACTACATCCCAC | 9 | TGCAGTCTAGGAAGTTGAC | 10 |
| | AC | | CA | |
| Col1a1 | GCTCCTCTTAGGGGCCACT | 11 | CCACGTCTCACCATTGGGG | 12 |
| Co13a1 | CTGTAACATGGAAACTGG | 13 | CCATAGCTGAACTGAAAAC | 14 |
| | GGAAA | | CAC | |
| Cpt1a | CTCCGCCTGAGCCATGAA | 15 | CACCAGTGATGATGCCATT | 16 |
| | CT | | | |
| Ccl2 | TTAAAAACCTGGATCGGA | 17 | GCATTAGCTTCAGATTTAC | 18 |
| | ACCAA | | GGGT | |
| Ccr2 | ATCCACGGCATACTATCAA | 19 | CAAGGCTCACCATCATCGT | 20 |
| | CATC | | AG | |
| Ddr2 | ATCACAGCCTCAAGTCAGT | 21 | TTCAGGTCATCGGGTTGCA | 22 |
| | GG | | C | |
| Fasn | GGAGGTGGTGATAGCCGG | 23 | TGGGTAATCCATAGAGCCC | 24 |
| | TAT | | AG | |
| G6pc | CGACTCGCTATCTCCAAGT | 25 | GTTGAACCAGTCTCCGACC | 26 |
| | GA | | A | |
| Il1b | GCAACTGTTCCTGAACTCA | 27 | ATCTTTTGGGGTCCGTCAA | 28 |
| | ACT | | CT | |
| Pck1 | ACACACACACATGCTCAC | 29 | ATCACCGCATAGTCTCTGA | 30 |
| | AC | | A | |
| Pdgfa | GAGGAAGCCGAGATACCC | 31 | TGCTGTGGATCTGACTTCG | 32 |
| | C | | AG | |
| Pdgfb | CATCCGCTCCTTTGATGAT | 33 | GTGCTCGGGTCATGTTCAA | 34 |
| | CTT | | GT | |
| Pdgfra | TCCATGCTAGACTCAGAAG | 35 | TCCCGGTGGACACAATTTT | 36 |
| | TC | | TC | |
| Ppargc1a | GAGAATGAGGCAAACTTG | 37 | TGCATGGTTCTGAGTGCTA | 38 |
| | CTAGCG | | AGACC | |
| Ppia | CCACTGTCGCTTTTCGCCG | 39 | TGCAAACAGCTCGAAGGA | 40 |
| | C | | GACGC | |
| Prkaa1 | TACTCAACCGGCAGAAGA | 41 | TCCTTTTCGTCCAACCTTCC | 42 |
| | TTCG | | A | |
| Prkaa2 | GACTTCCTTCACAGCCTCA | 43 | CGAGCGACTATCAAAGAC | 44 |
| | TC | | ATACG | |
| Ripk1 | GAAGACAGACCTAGACAG | 45 | CCAGTAGCTTCACCACTCG | 46 |
| | CGG | | AC | |
| Ripk3 | TCTGTCAAGTTATGGCCTA | 47 | GGAACACGACTCCGAACCC | 48 |
| | CTGG | | | |
| Srebf1 | TGACCCGGCTATTCCGTGA | 49 | CTGGGCTGAGCAATACAGT | 50 |
| | TC | | | |
| Tgfb | CTCCCGTGGCTTCTAGTGC | 51 | GCCTTAGTTTGGACAGGAT | 52 |
| | CTG | | | |
| Timp1 | GCAACTCGGACCTGGTCAT | 53 | CGGCCCGTGATGAGAAACT | 54 |
| | AA | | | |
| Tnfa | CCCTCACACTCAGATCATC | 55 | GCTACGACGTGGGCTACA | 56 |
| | TTCT | | | |

### Caspase-6 Knockdown

HepG2 cells were transfected with negative control siRNA (Life Technologies, Cat. AM4611) or casp6 siRNA (Life Technologies, Cat. 4390824) using Lipofectamine RNAiMAX Transfection Reagent (Life Technologies, Cat. 13778075) in antibiotic-free medium.

### Transfection

1 μg DNA for each plasmid was used to transfect HEK239T cells with Lipofectamine 3000 (Life Technologies), according the manufacturer's instruction. 24 hrs later, cells were harvested and subject to Western blot analysis.

### Cell Viability Assay

Cells were incubated with medium containing 1/10 Cell Counting Kit-8 (CCK-8) (Biomake, Cat. B34302) at 37° C. for 30 min. Absorbance was measured at 450 nm.

### In Vitro Cleavage Assay

Immunoprecipitated Bax-HA or Bid-HA, recombinant Bid (R&D Systems, Cat. 846-BD-50), recombinant Caspase-6 (Origene, Cat. TP760261) was incubated with 1 unit active caspase-3, -6, -7, -8 or -9 (Enzo Life Sciences) in a 25 μl reaction containing 50 mM HEPES (pH 7.5), 3 mM EDTA, 150 mM NaCl, 0.005% Tween-20 and 10 mM DTT at 37° C. for 60 min(41).

### N-Terminal Sequencing

Product from the in vitro cleavage assay was separated by NuPAGE protein gel. Following Coomassie Brilliant Blue staining, bands were cut and subject to Edman Degradation, which was performed by Bio-Synthesis Inc.

### In Vitro Kinase Assay

Immunoprecipitated Myc-Casp6 WT or recombinant Caspase-6 (Origene, Cat. TP760261) was subject to in vitro kinase assay with recombinant AMPKα1β1γ1 (Signalchem, Cat. P48-10H-10)/AMPKα2β1γ1 (Signalchem, Cat. P48-10H-05), kinase dilution buffer VII (SignalChem, Cat. K27-09), 100 μM ATP (SignalChem, Cat. A50-09) and 100 μM AMP (SignalChem, Cat. A46-09). After 15 min incubation in 30° C. water bath, reaction was stopped by adding SDS loading buffer and heating at 95° C. Phospho-Caspase-6 Ser²⁵⁷ antibody (Life Technologies, Cat. PA5-12557) was used to detect phosphorylated Caspase-6.

### Site-Directed Mutagenesis

Site-directed mutagenesis was performed as previously described(11). Myc-Casp6 S²⁵⁷A, S²⁵⁷D, S²⁵⁷E plasmids were generated using Kapa Hifi DNA polymerase with 0.5 ng Myc-Casp6 WT as template DNA in 50 ul reaction. Template DNA was digested by DpnI (NEB). Stellar competent cell (Takara) was used for transformation and plasmids amplification. Plasmid was purified with Wizard Plus SV Minipreps DNA purification system (Promega). Mutation was confirmed by DNA sequencing (Eton Bioscience).

### Quantification and Statistical Analysis

All data in animal studies are shown as mean±SEM, while data from in vitro studies are shown as mean±SD. Replicates are indicated in figure legends. N represents the number of experimental replicates. F-test was performed to determine the equality of variance. When comparing two groups, statistical analysis was performed using a two-tailed Student's t-test, except when the f-test suggested that variances are statistically different. For analysis of more than two groups, we used analysis of variance (ANOVA) to determine equality of variance. Comparisons between groups were performed with Tukey-Krammer post-hoc analysis. For all tests, P<0.05 was considered statistically significant (11).

### Example 2

### Liver-Specific AMPK Knockout Exaggerates Liver Damage in NASH

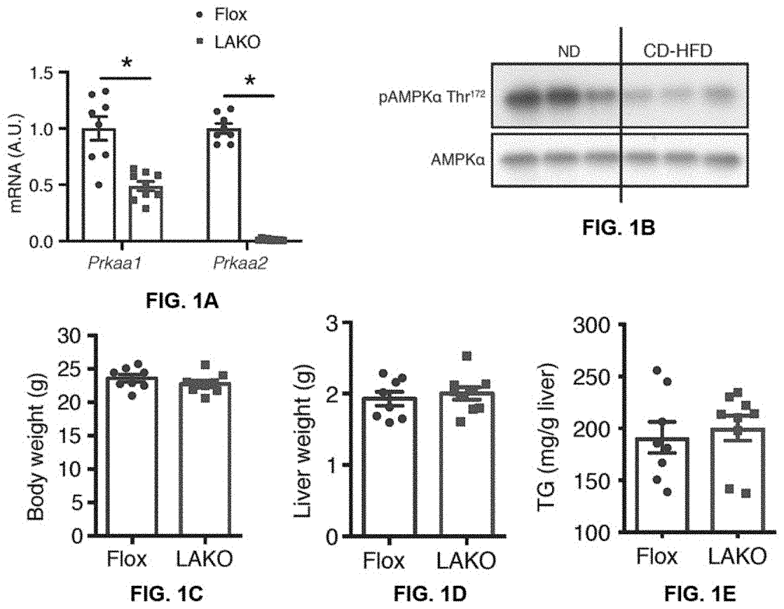

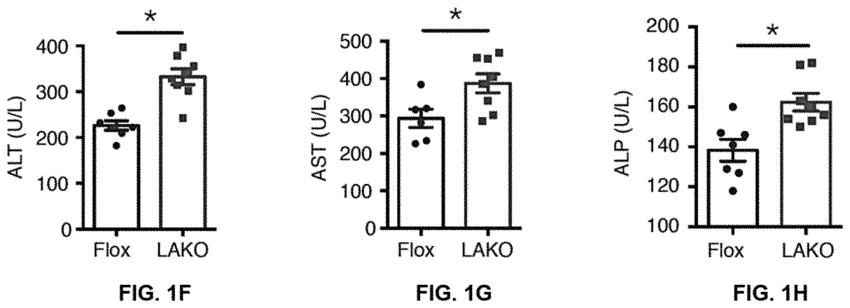

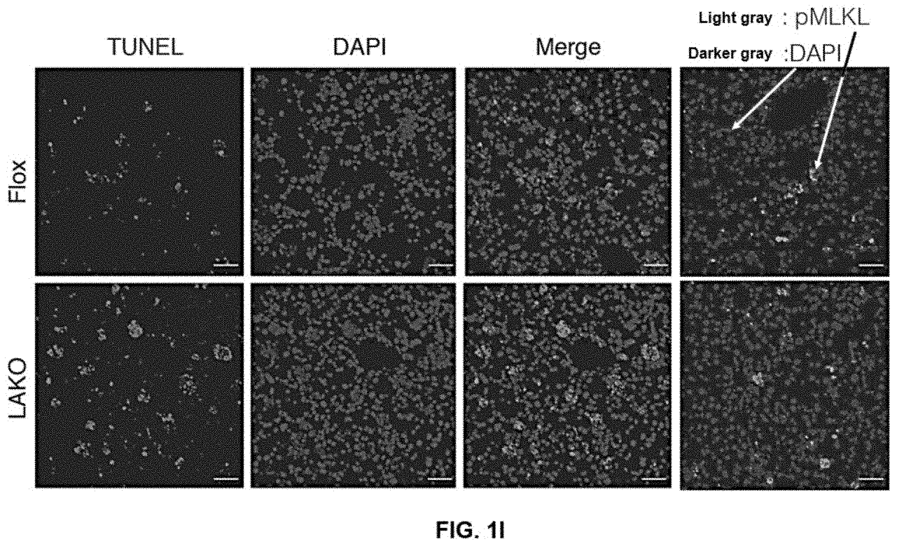

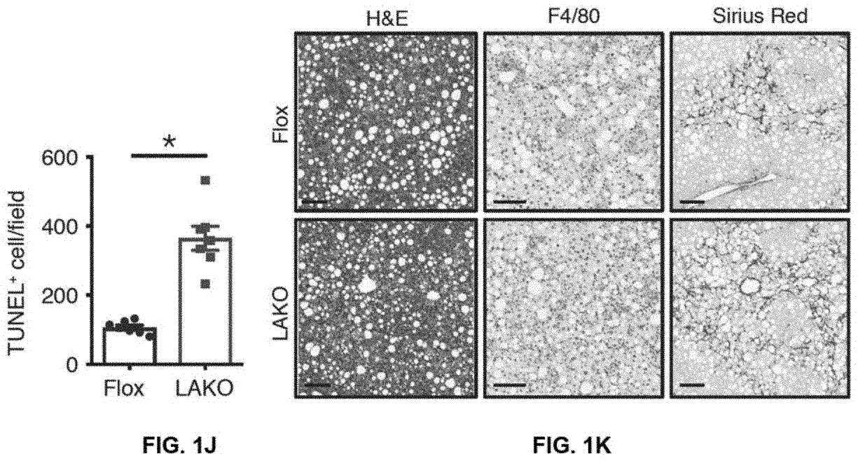

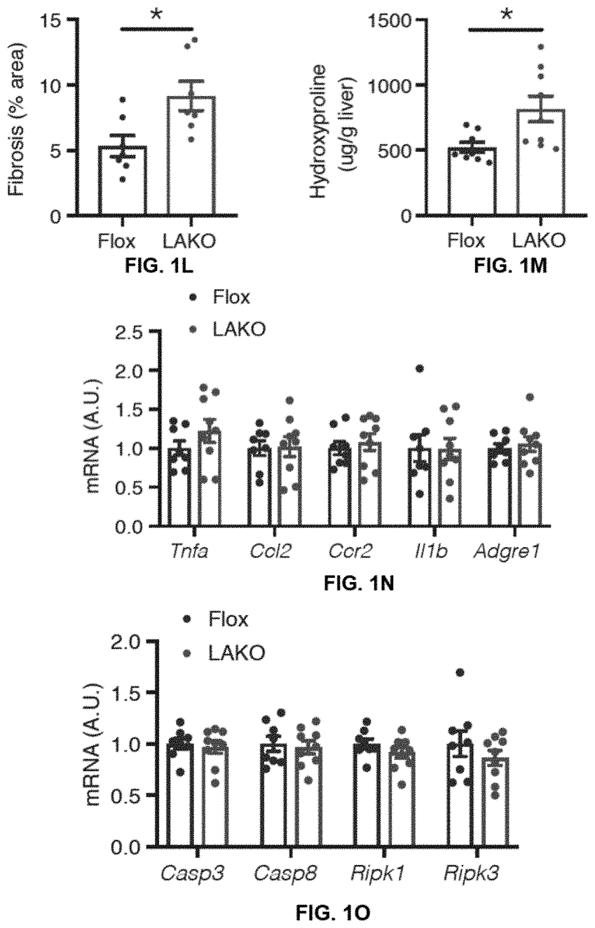

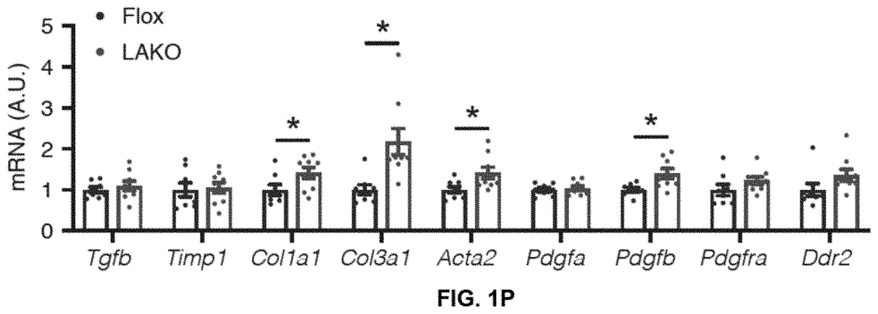

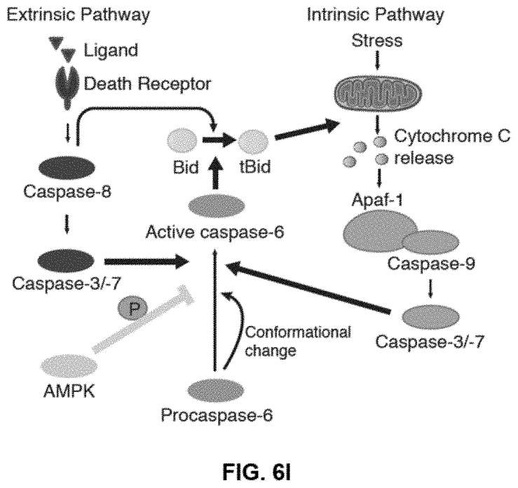

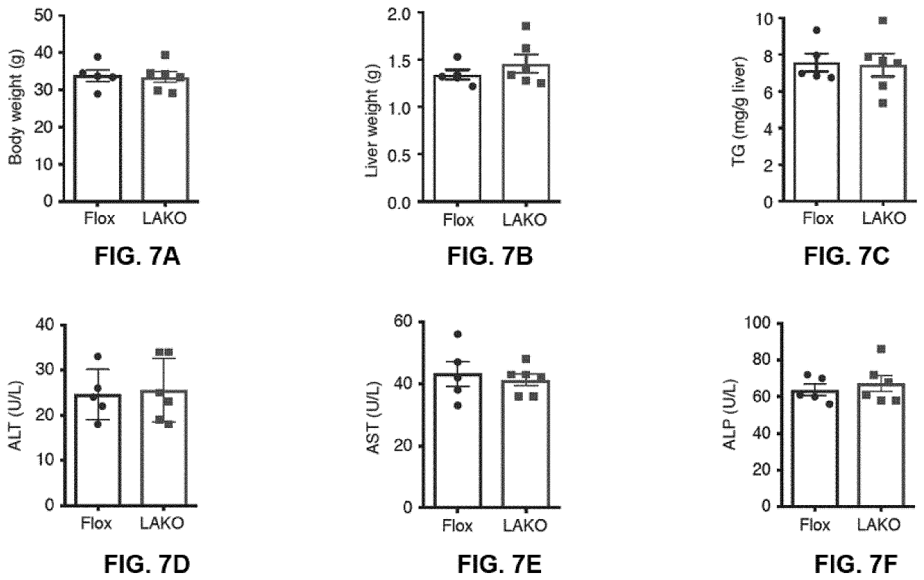

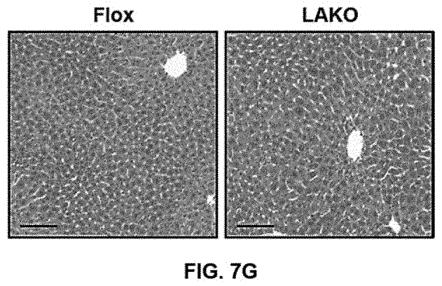

Hepatic AMPK activity is suppressed in diet-induced NAFL (9, 13). Although AMPK activation attenuates steatosis, loss of AMPK does not induce steatosis (13). Moreover, the role of AMPK in the pathogenesis of NASH remains uncertain. Liver-specific AMPKα1/α2 (Prkaa1/Prkaa2) double knockout (LAKO) mice that are devoid of hepatocyte expression of AMPKα1 and α2, the catalytic subunits of AMPK were generated (FIG. 1A). Liver-specific AMPK ablation did not affect body weight, liver weight, or triglycerides (TG) in mice fed normal chow diet (ND) (FIGS. 7A-7C). ND-fed LAKO mice had normal serum alanine aminotransferase (ALT), aspartate aminotransferase (AST), alkaline phosphatase (ALP) activities and liver morphology (FIGS. 7D-7G).

Flox and LAKO mice were fed with a choline-deficient, high fat diet (CD-HFD) to rapidly induce hepatic steatosis, liver damage, and fibrosis, characteristics of NASH (16). CD-HFD decreased AMPK Thr¹⁷² phosphorylation in livers of C57BL/6J mice, indicating repression of AMPK activity (FIG. 1B). LAKO mice were identical to Flox mice with respect to body weight, liver weight or hepatic TG (FIGS. 1C-1E). However, a significant increase of serum ALT, AST and ALP activities suggested exaggerated liver damage in CD-HFD fed LAKO mice (FIGS. 1F-1H). Increased liver TUNEL staining demonstrated that AMPK knockout substantially increased the number of apoptotic cells, without affecting necroptotic cells identified by staining with phosphorylated Mixed Lineage Kinase Domain-Like Protein (phospho-MLKL) (FIGS. 1I and 1J). LAKO mice showed no changes in liver macrophage infiltration as evidenced by similar macrophage marker F4/80 staining in Flox and LAKO mice (FIG. 1K).

Nonetheless, hepatic fibrosis as measured by Sirius Red staining and abundance of hydroxyproline were increased in LAKO mice, correlating with enhanced scarring from exaggerated liver damage (FIGS. 1K-1M). LAKO did not affect the expression of the macrophage marker Adhesion G Protein-Coupled Receptor E1 (Adgre1, F4/80), the chemotactic cytokine C-C Motif Chemokine Ligand 2 (Ccl2) and its receptor Ccr2, or pro-inflammatory cytokines Tumor Necrosis Factor alpha (Tnfa) and Interleukin-1beta (Il1b) (FIG. 1N). Although LAKO increased cell death and liver damage, the expression of cell death mediators Caspase-3 (Casp3), Casp8, Receptor-Interacting Serine/Threonine Protein Kinase 1 (Ripk1) and Ripk3 was not affected (FIG. 1O). Consistent with increased fibrosis, LAKO increased the expression of the fibrosis marker gene Actin alpha2 (Acta2), collagen genes Collagen Type I alpha1 (Col1a1) and Col3a1, as well as hepatic stellate cell activating growth factor Platelet Derived Growth Factor Subunit B (Pdgfb) (FIG. 1P). LAKO mice showed no differences in the expression of Transforming Growth Factor beta (Tgfb), the major macrophage-derived fibrogenic cytokine, consistent with similar macrophage infiltration in Flox and LAKO mice. Likewise, LAKO mice showed no difference in growth factor Pdgfa and receptor Pdgfra expression, or matrix remodeling genes Tissue Inhibitor Matrix Metalloproteinase 1 (Timp1) and Discoidin Domain Receptor Tyrosine Kinase 2 (Ddr2) (FIG. 1P).

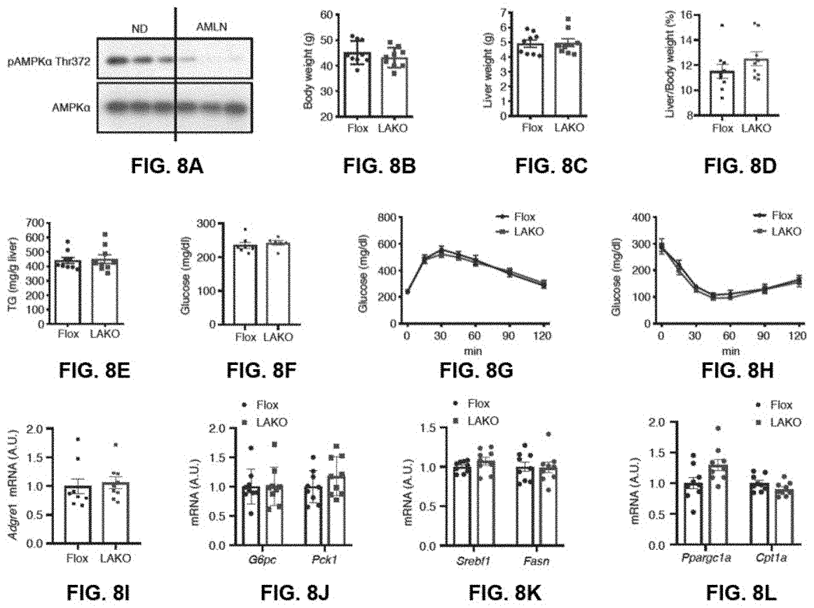

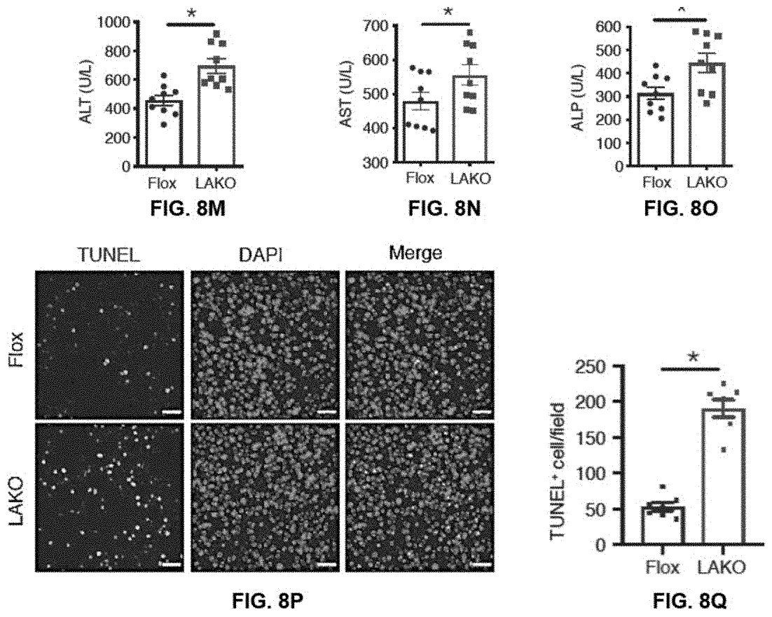

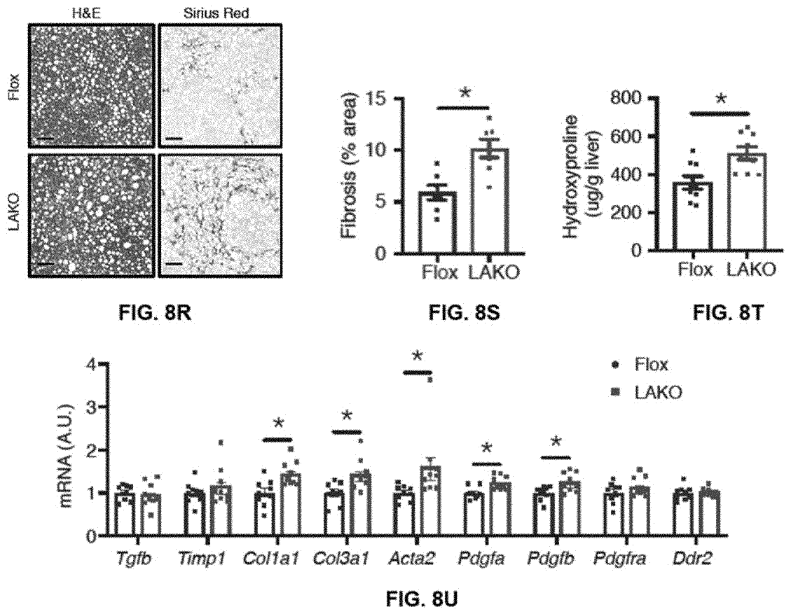

Mice were also fed with the Amylin (AMLN) diet, used to mimic human NASH in pre-clinical studies (2, 17). AMLN also repressed AMPK activation in C57BL/6J mice (FIG. 8A). LAKO had no effect on body weight, liver weight, liver to body weight ratio, hepatic TG, fasting glucose, glucose tolerance, or insulin resistance (FIGS. 8B-8H). LAKO did not affect the expression of Adgre1 , gluconeogenic genes Glucose-6-Phosphatase Catalytic Subunit (G6pc) and Phosphoenolpyruvate Carboxykinase 1 (Pck1), lipogenic genes Sterol Regulatory Element Binding Transcription Factor 1 (Srebf1) and Fatty Acid Synthase (Fasn), or mitochondria and lipid oxidation regulation genes Peroxisome Proliferator Activated Receptor Gamma Coactivator 1 alpha (Ppargc1a) and Carnitine Palmitoyltransferase 1a (Cpt1a) (FIGS. 8I-8L) in AMLN-fed mice. However, LAKO significantly increased serum ALT, AST and ALP activities, suggesting enhanced liver damage (FIGS. 8M-8O). LAKO mice had increased number of apoptotic liver cells (FIGS. 8P and 8Q). Furthermore, exaggerated liver damage in LAKO mice led to increased fibrosis (FIGS. 8R-8T). Similar to the results from CD-HPD-fed mice, LAKO increased the expression of fibrosis markers Acta2, Col1a1 and Col3a1 and fibrogenic growth factors Pdgfa and Pdgfb, with no effect on Tgfb, Timp1, Pdgfra, or Ddr2 in AMLN-fed mice (FIG. 8U). Thus, while LAKO did not further exaggerate steatosis or inflammation during NASH, hepatocellular death and fibrosis were enhanced.

### Example 3

### AMPK Deficiency Increases Caspase-6 Activation to Promote Liver Damage in NASH

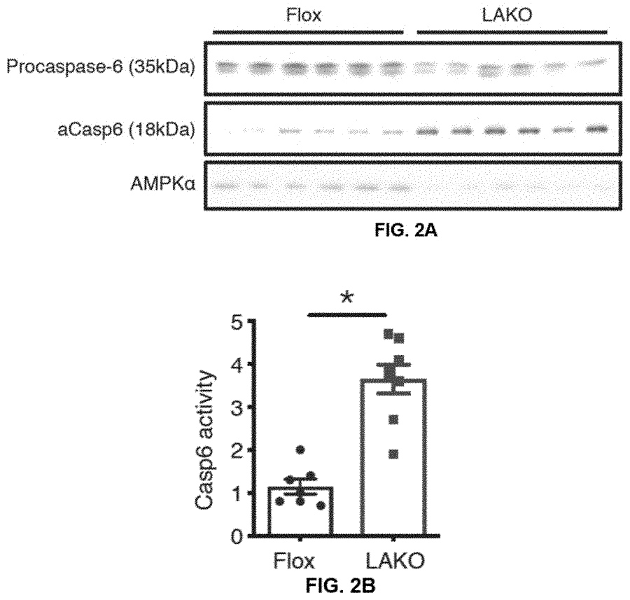

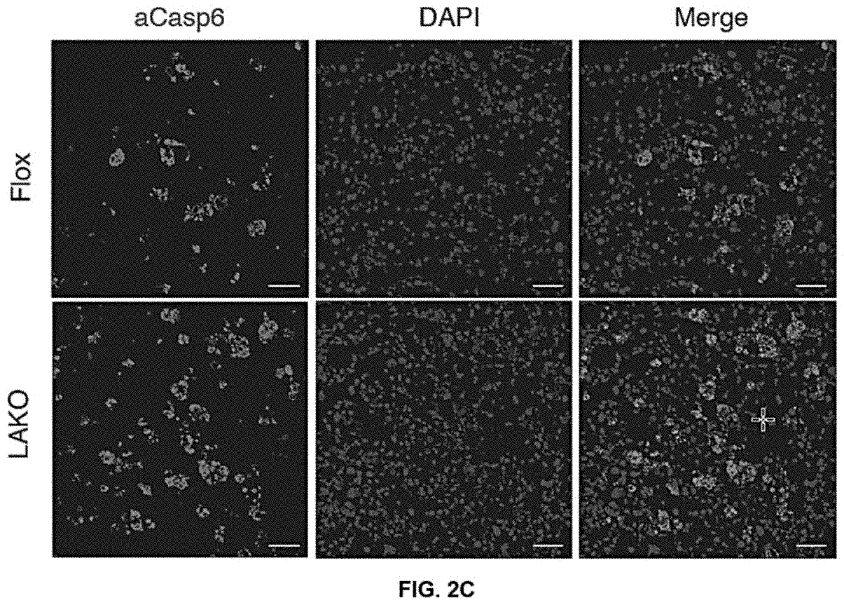

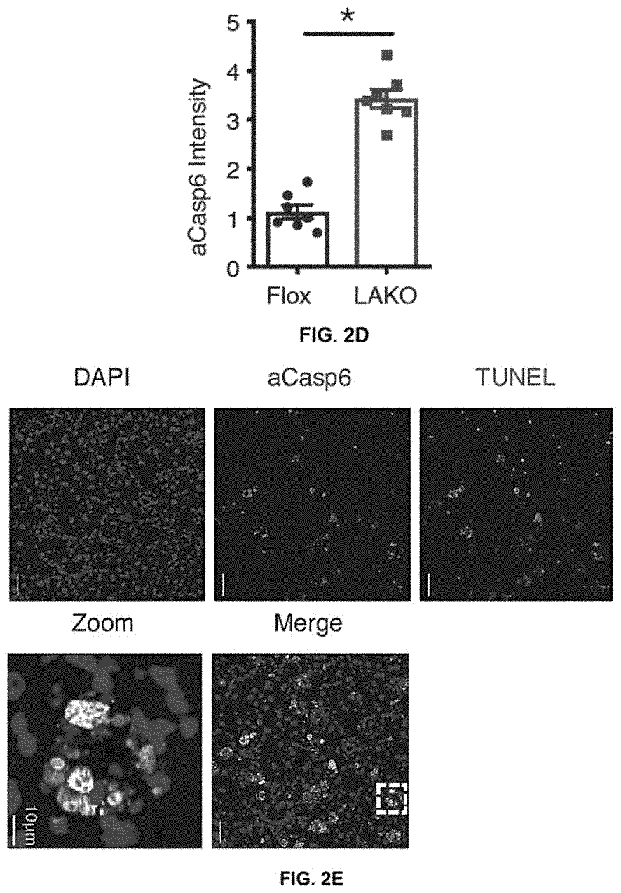

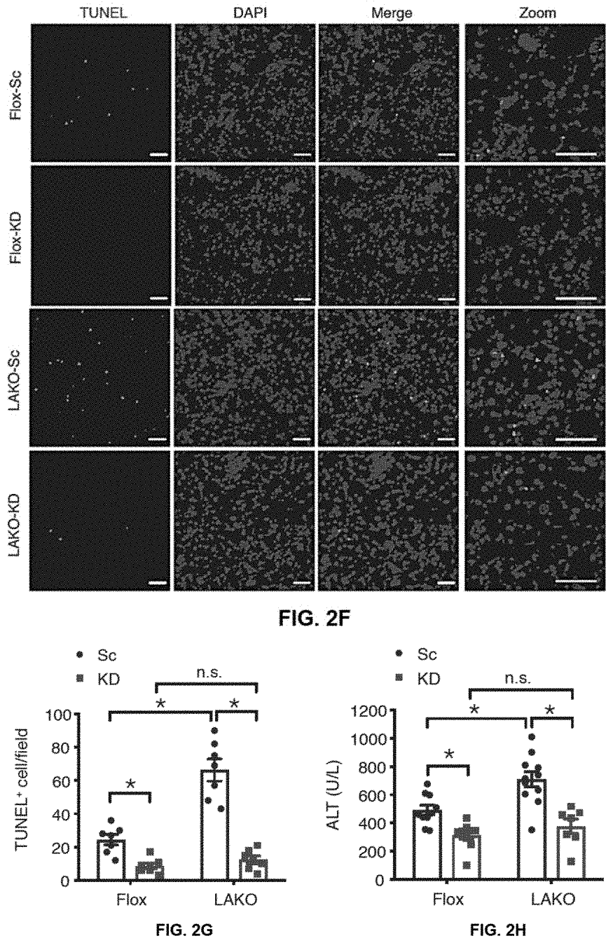

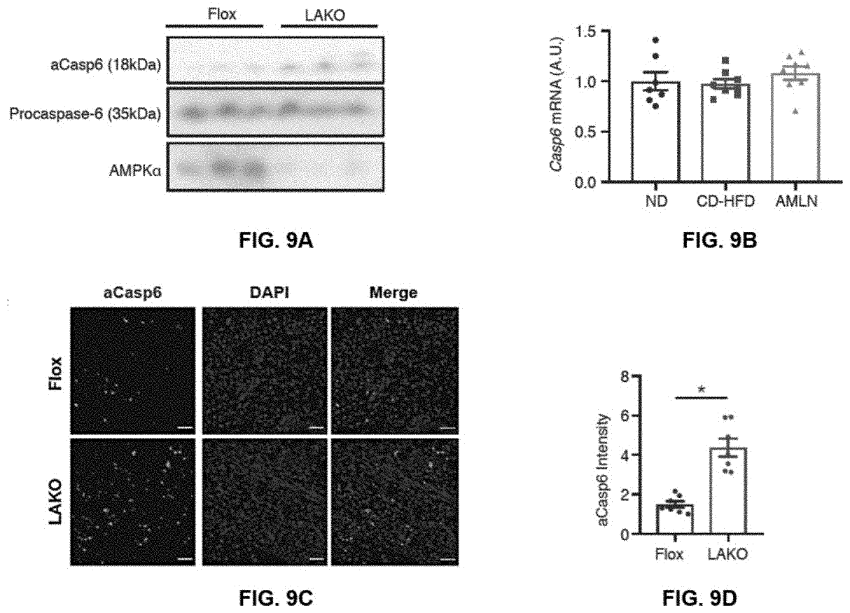

To investigate the mechanism of exacerbated liver damage in NASH, proapoptotic caspases was focused on, as necroptosis was not affected by LAKO. Cleavage of procaspase-6 and caspase-6 activity were increased in livers of LAKO mice on both CD-HFD (FIGS. 2A and 2B) and AMLN diets (FIG. 9A). Casp6 mRNA was not regulated by either diet (FIG. 9B). LAKO significantly increased active caspase-6 (aCasp6) in livers of mice on CD-HFD (FIGS. 2C and 2D) or AMLN diet (FIGS. 9C and 9D). Co-staining of TUNEL and aCasp6 revealed TUNEL-stained nuclei located within cells with aCasp6 (FIG. 2E), correlating caspase-6 activation with hepatocellular death in NASH.

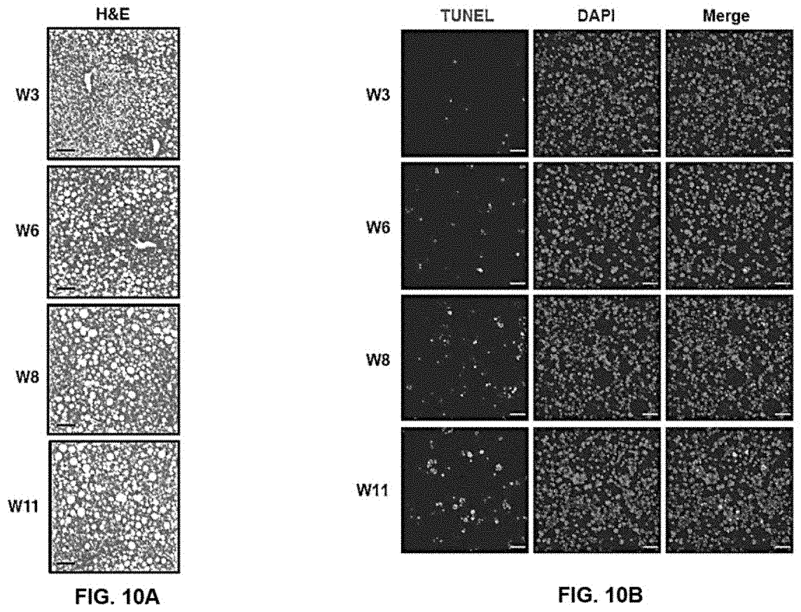

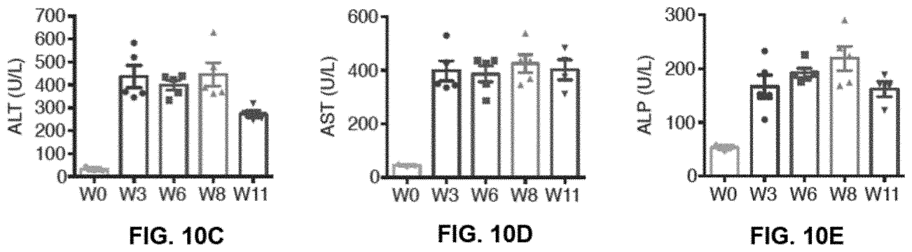

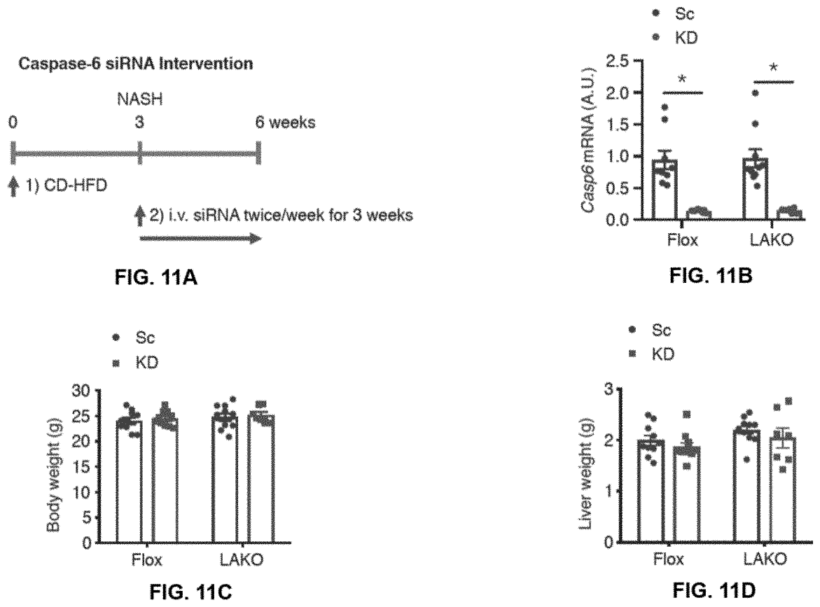

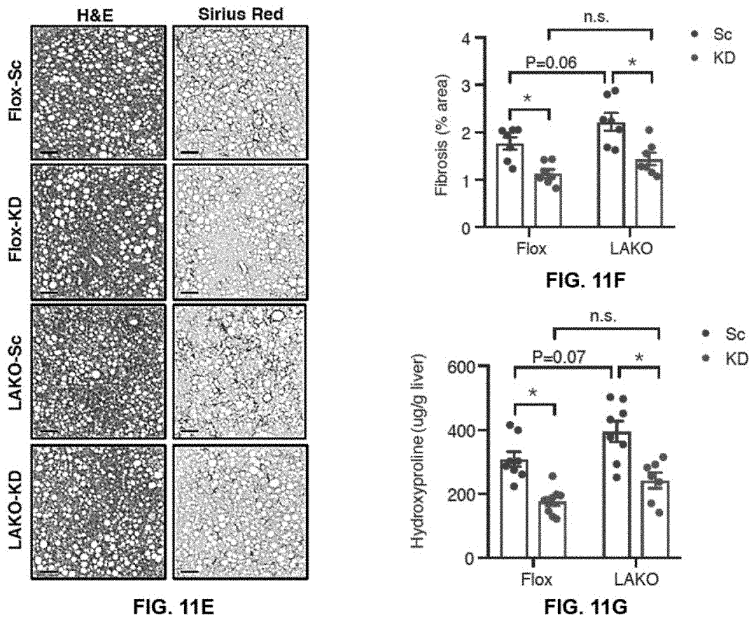

The temporal relationship of caspase-6 activation and NASH development was examined Symptoms of NASH were apparent by 3 weeks of CD-HFD feeding, as evidenced by development of steatosis, TUNEL staining, and increased activities of ALT, AST, and ALP in serum. After 6 weeks, the mice developed extensive steatosis and significant TUNEL staining, indicating well-established NASH (FIGS. 10A-10E). After 3 weeks CD-HFD to develop NASH, Flox and LAKO mice intravenously injected with control or caspase-6 siRNA for 3 weeks during continuous CD-HFD (FIG. 11A). Caspase-6 siRNA reduced hepatic Casp6 mRNA by more than 80%. LAKO did not affect Casp6 expression (FIG. 11B). Caspase-6 depletion did not affect body or liver weight in CD-HFD-fed mice of either genotype (FIGS. 11C and 11D). However, caspase-6 depletion reduced the number of apoptotic hepatocytes and serum ALT activity in both Flox and LAKO mice to a similar extent (FIGS. 2F-2H). Depletion of caspase-6 significantly attenuated fibrosis in Flox and LAKO mice and nullified the deleterious effects of LAKO (FIGS. 11E-11G).

### Example 4

### Caspase-6 is Activated in Murine and Human NASH

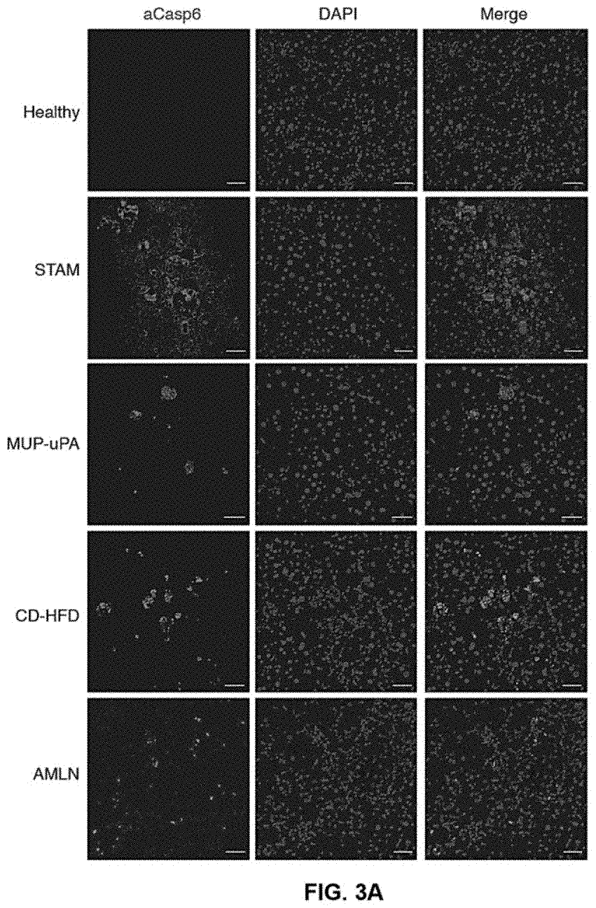

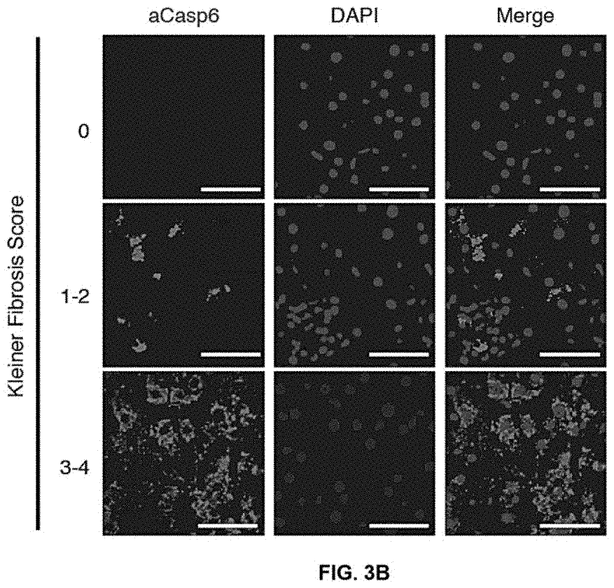

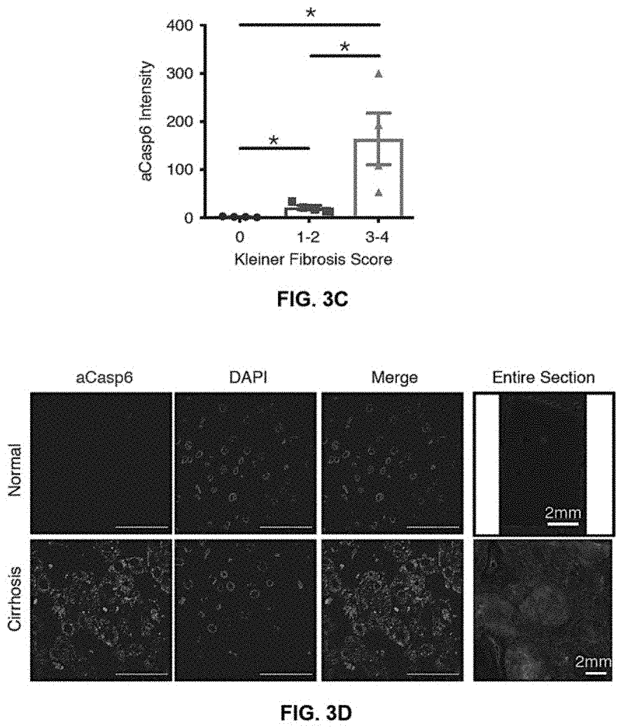

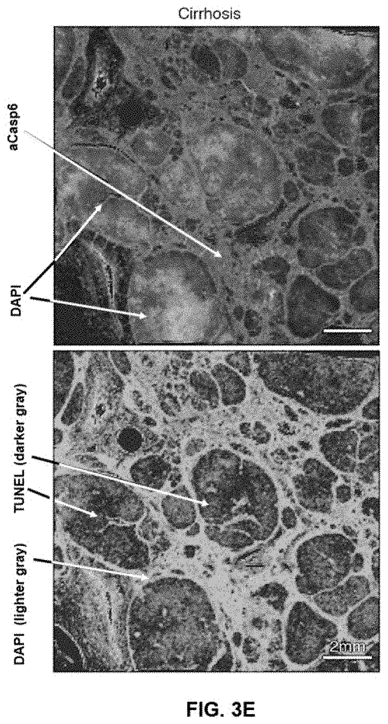

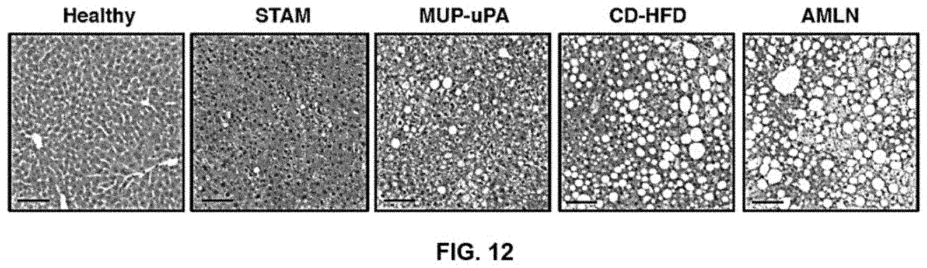

Since depleting caspase-6 attenuated liver damage in CD-HFD-induced NASH, we examined whether the activation of caspase-6 might occur in other NASH models, including HFD-fed streptozotocin-administered neonatal mice (18), HFD-fed major urinary protein-urokinase-type plasminogen activator (MUP-uPA) transgenic mice (19), or even human NASH. Caspase-6 was activated in livers of all mouse NASH models, but not in healthy livers (FIG. 3A). The presence of NASH was validated by H&E staining (FIG. 12A). To determine if caspase-6 is activated in human NASH, aCasp6 was blindly assessed in liver sections of NASH patients, in whom liver status had been diagnosed. Caspase-6 was activated in livers from patients with NASH and cirrhosis (FIGS. 3B-3D). Active caspase-6 significantly increased with Kleiner fibrosis score, and positively correlated with severity of NASH (FIGS. 3B and 3C). Furthermore, while sections from normal livers had almost no active caspase-6 staining, the degree of active caspase-6 was increased in cirrhosis (FIG. 3D). Moreover, whole section scanning showed that both active caspase-6 and TUNEL staining were located within the cirrhotic nodule, indicating that caspase-6 might activate apoptosis of hepatocytes in human cirrhosis (FIG. 3E).

### Example 5

### An AMPK Agonist and a Caspase-6 Inhibitor Therapeutically Improve Liver Damage

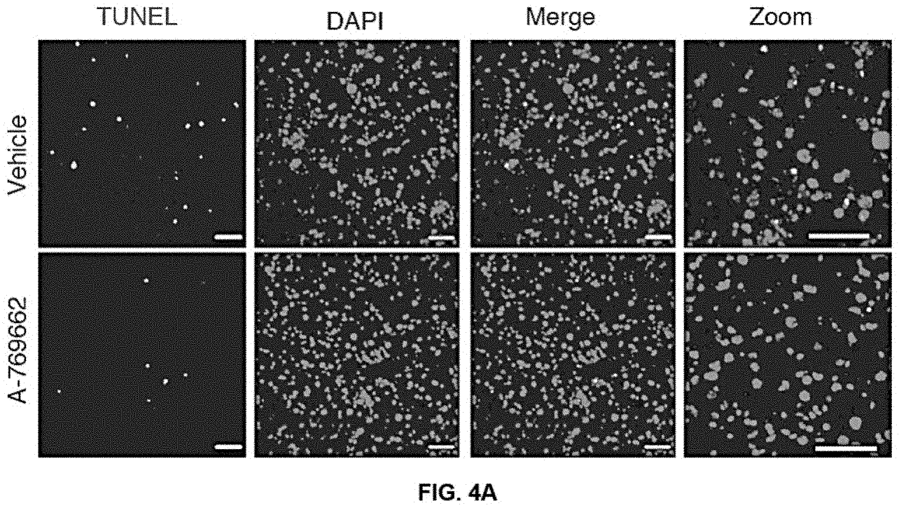

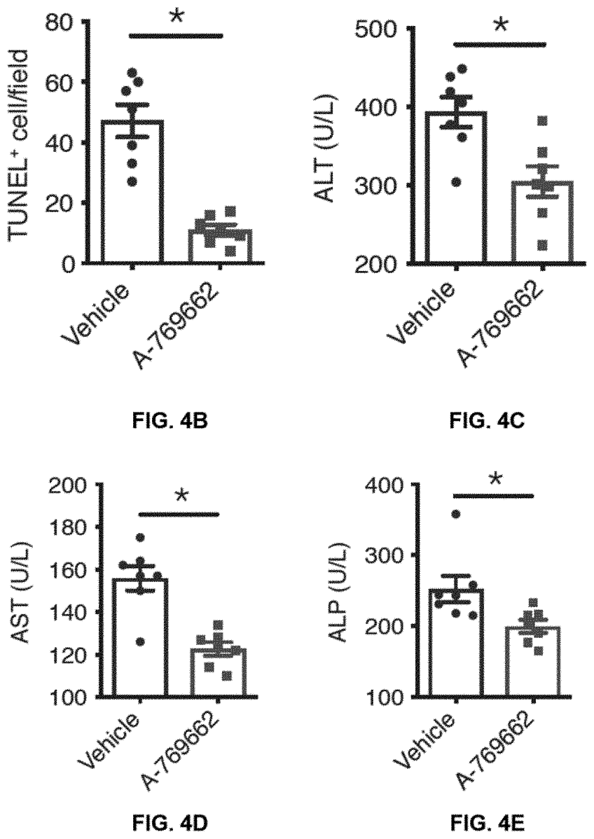

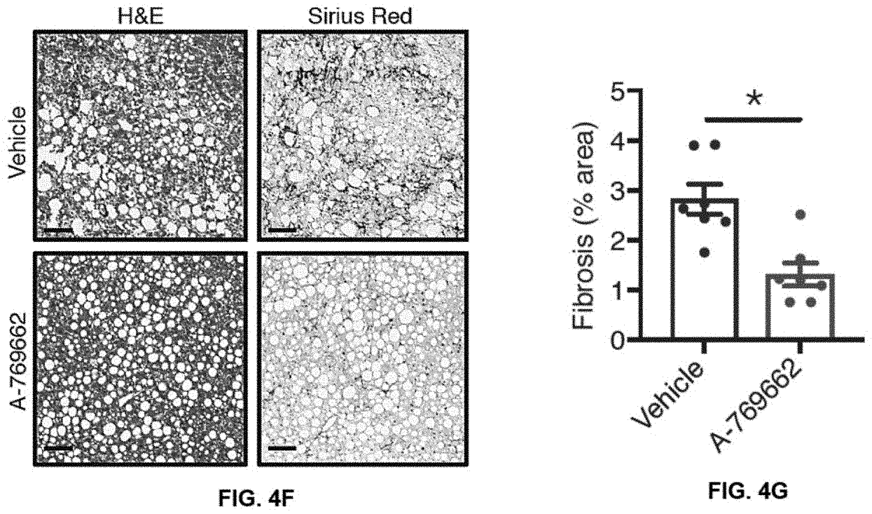

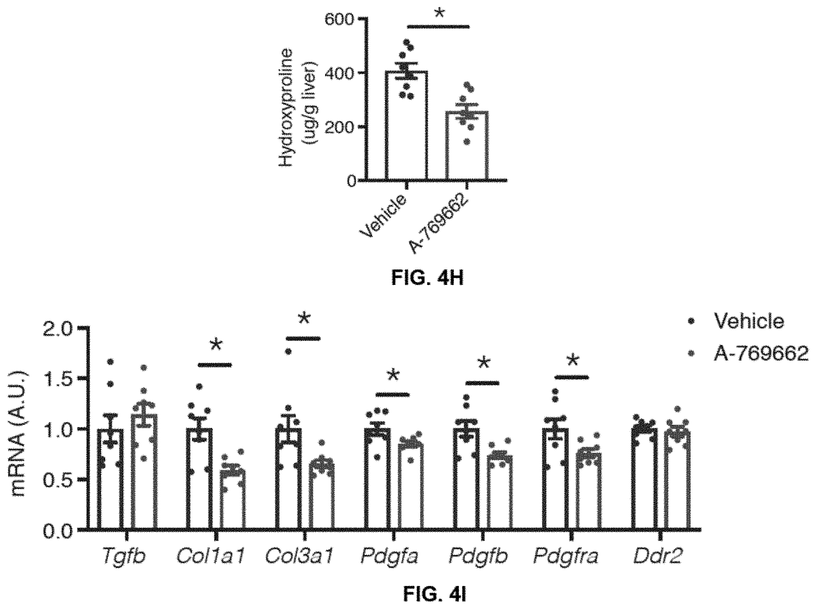

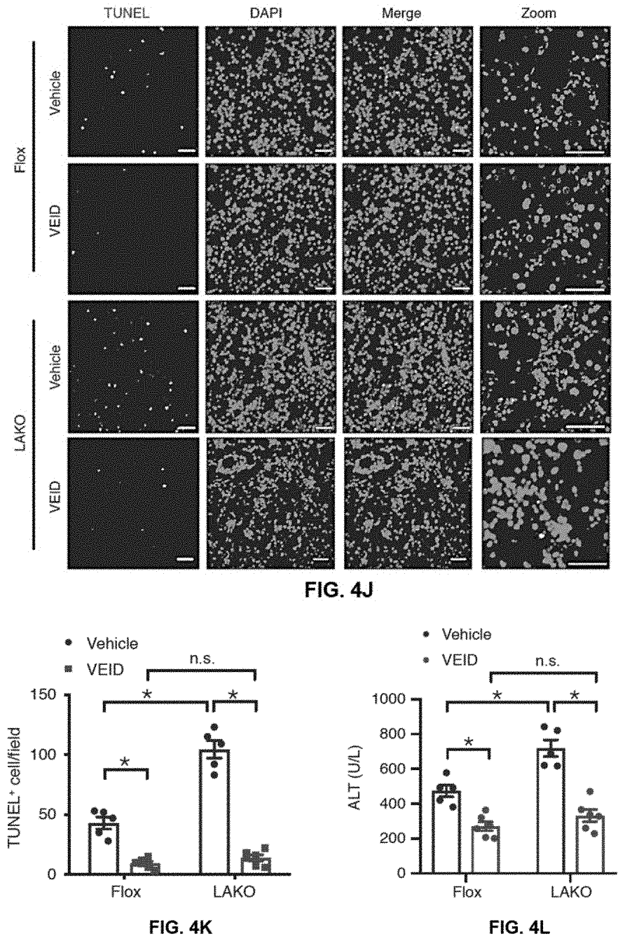

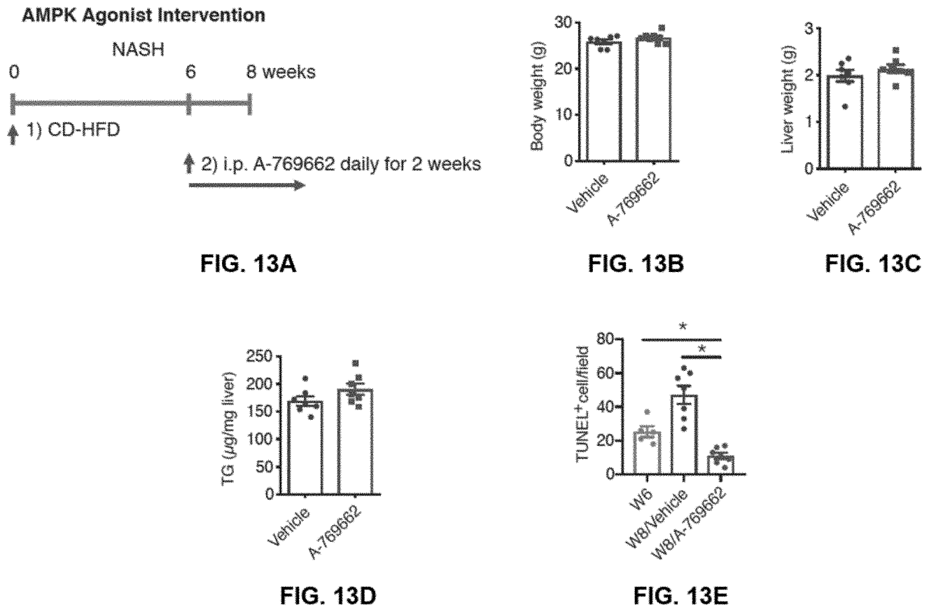

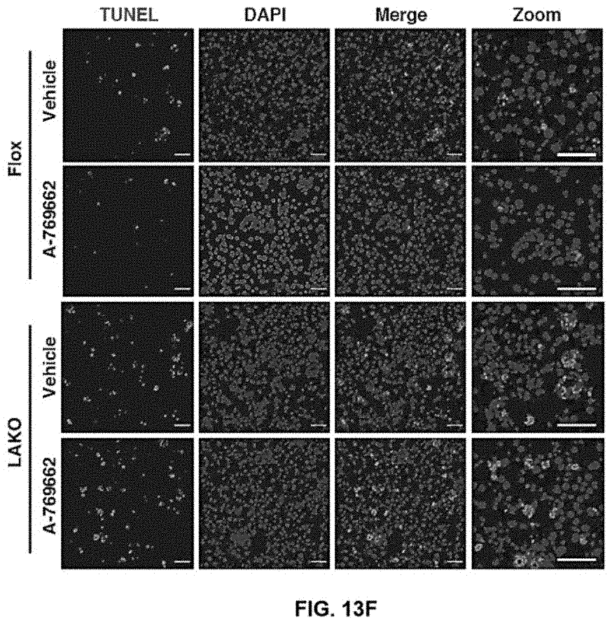

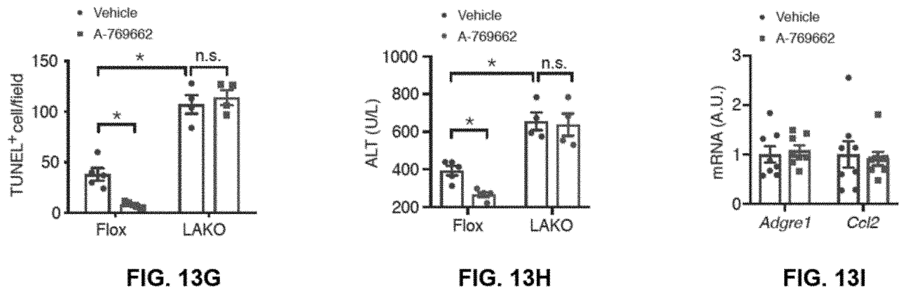

To test the potential of AMPK as a therapeutic target, C57BL/6J mice were fed with CD-HFD for 6 weeks to establish NASH, and then these mice were intraperitoneally injected with vehicle or AMPK agonist (A-769662) for 2 weeks while continuing CD-HFD (FIG. S13A). In mice, AMPKβ1 is expressed in liver but not skeletal muscle (20). Thus, as an AMPKβ1 agonist, A-769662 activates AMPK in liver but not skeletal muscle. Previous study showed that injection of 30 mg/kg A-769662 twice per day for 7 days reduced hepatic triglycerides in C57BL/6J mice fed 45% HFD (13). In this study, injection of 25 mg/kg A-769662 once per day for two weeks in CD-HFD fed mice had no effect on body weight, liver weight or hepatic TG (FIGS. 13B-13D), but significantly reduced the number of apoptotic cells (FIGS. 4A and 4B; and FIG. 13E) and improved liver damage (FIGS. 4C-4E). A-769662 injection did not decrease the number of apoptotic cells nor reduce serum ALT activity in LAKO mice, indicating that A-769662 specifically targeted AMPK in hepatocytes to improve liver damage (FIGS. 13F-13H). A-769662 attenuated hepatic fibrosis (FIGS. 4F-4H), and significantly reduced the expression of fibrotic genes Col1a1, Col3a1, Pdgfa, Pdgfb, Pdgfra, without effect on Tgfb, Ddr2 or inflammation marker genes Adgre1, Ccl2 (FIG. 4I and FIG. 13I).

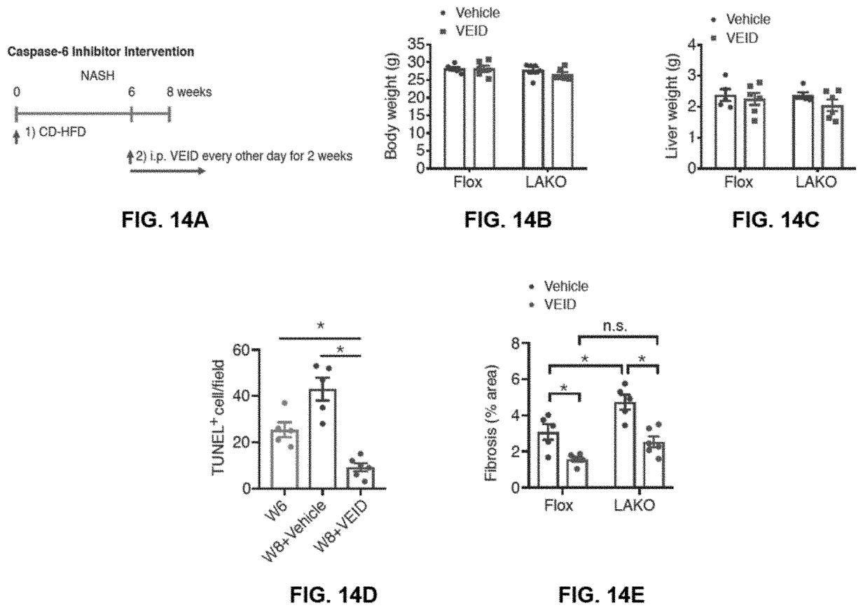

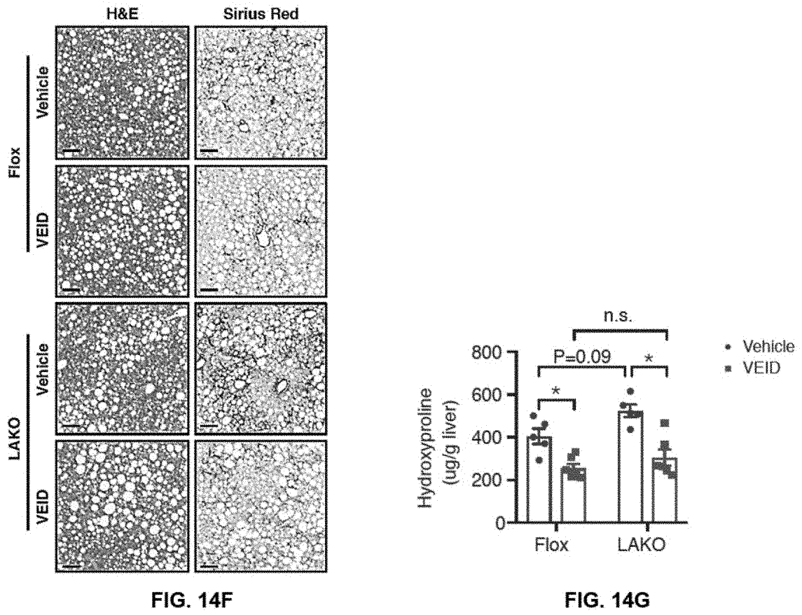

To examine if caspase-6 inhibition might also improve liver damage and decrease the effects of AMPK deficiency, Flox and LAKO mice were fed with CD-HFD for 6 weeks to establish NASH, and then intraperitoneally injected vehicle or the caspase-6 inhibitor Z-VEID-FMK (VEID) (SEQ ID NO: 71) for two weeks while continuing CD-HFD (FIG. 14A). VEID (SEQ ID NO: 71) did not affect body or liver weight (FIGS. 14B and 14C), but significantly reduced the number of apoptotic hepatocytes and decreased serum ALT activity in both Flox and LAKO mice (FIGS. 4J-4L; and FIG. 14D). Of note, VEID (SEQ ID NO: 71) abrogated the difference in liver damage between Flox and LAKO mice. VEID (SEQ ID NO: 71) reduced liver fibrosis in both Flox and LAKO mice and abolished the effect of AMPK deficiency (FIGS. 14E-14G), further suggesting that AMPK-caspase-6 axis critically controls liver damage.

### Example 6

### AMPK Phosphorylates Procaspase-6 to Inhibit its Cleavage and Activation

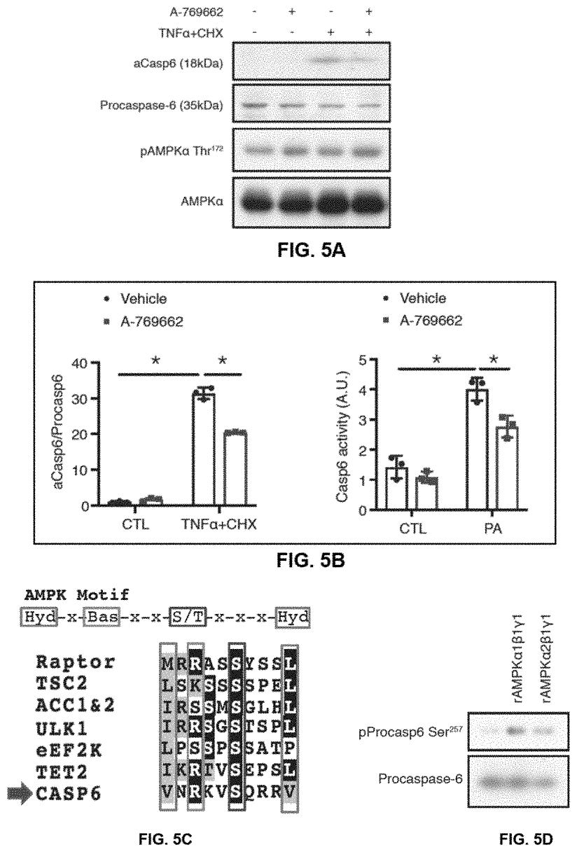

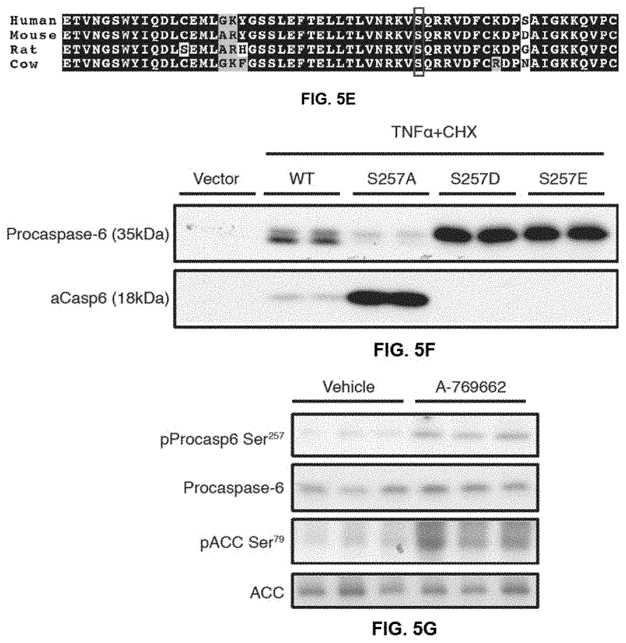

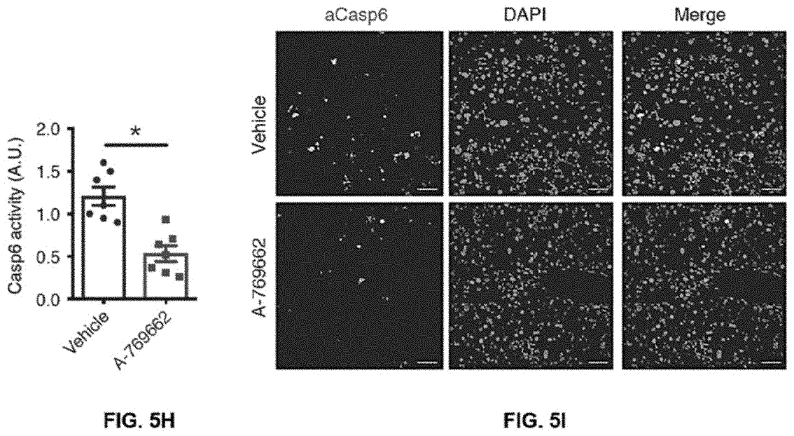

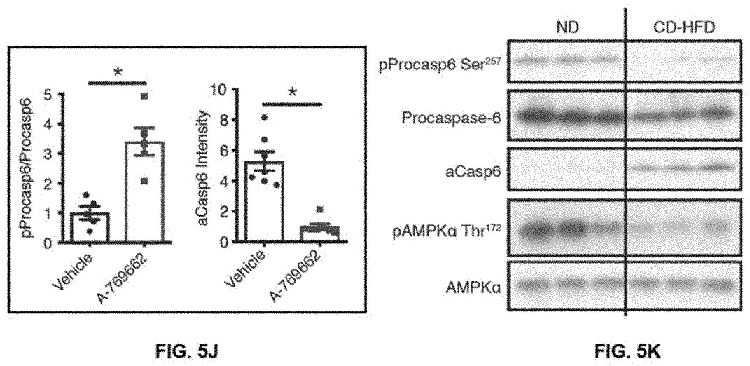

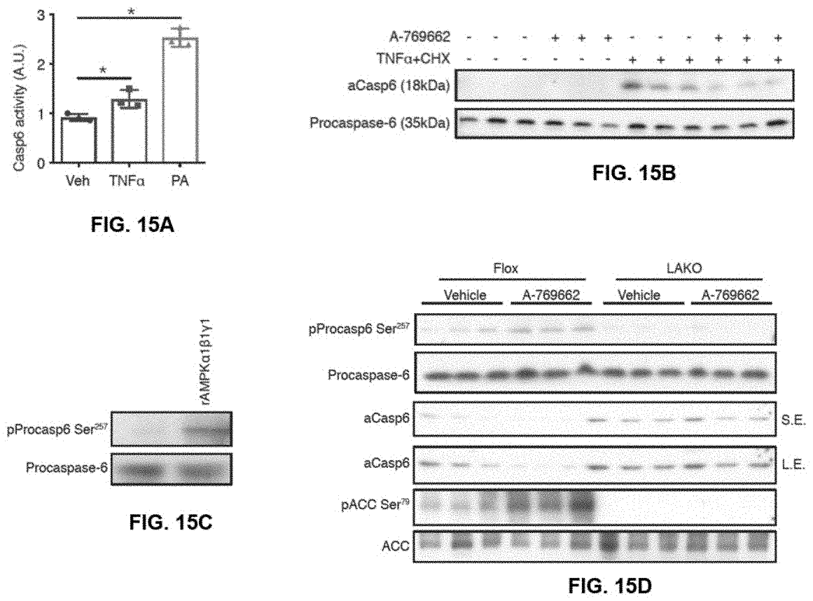

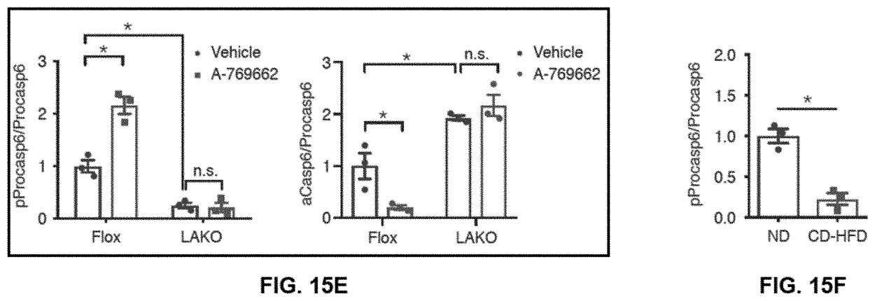

To explore the regulation of caspase-6, primary hepatocytes were treated with TNFα and palmitic acid (PA) to mimic inflammation and lipotoxicity-induced hepatocellular death. Both induced caspase-6 activation (FIG. 15A). To determine if the AMPK agonist directly inhibited procaspase-6 cleavage in a cell autonomous manner, primary hepatocytes or HepG2 cells were treated with A-769662, and then with TNFα and cycloheximide (CHX) to induce procaspase-6 cleavage. In both cells, A-769662 significantly inhibited procaspase-6 cleavage (FIG. 5A; and FIG. 15B). The induction of caspase-6 activity by PA was also inhibited in cells pre-treated with A-769662 (FIG. 5B). In silico analysis revealed one AMPK substrate motif site on procaspase-6 Ser²⁵⁷ (FIG. 5C) (21). Procaspase-6 Ser²⁵⁷ is phosphorylated by AMPK-Related Protein Kinase 5 (ARKS) in colorectal adenocarcinoma cells (22). Phosphorylation of Ser²⁵⁷ represses procaspase-6 cleavage and activation (23). Both recombinant AMPKα1 and AMPKα2 directly phosphorylated procaspase-6 at Ser²⁵⁷ in vitro (FIG. 5D; and FIG. 15C). The Ser²⁵⁷ site and the surrounding sequence in caspase-6 are conserved across species (FIG. 5E). WT procaspase-6, or its S²⁵⁷A non-phospho-mimetic mutant, or S²⁵⁷D/S²⁵⁷E phospho-mimetic mutants were overexpressed in HEK293T cells, and these cells were subsequently treated with low dose of TNFα and CHX to induce procaspase-6 cleavage. The S²⁵⁷A mutant was more sensitive to cleavage, while both the S²⁵⁷D and S²⁵⁷E mutants were completely resistant (FIG. 5F). Moreover, A-769662 significantly increased procaspase-6 Ser²⁵⁷ phosphorylation (FIG. 5G), and decreased caspase-6 activity in vivo (FIG. 5H). AMPK activation decreased aCasp6 in CD-HFD-induced NASH (FIGS. 5I and 5J). Analysis of liver lysates from CD-HFD-fed Flox and LAKO mice administered vehicle or A-769662 revealed that A-769662 significantly increased procaspase-6 phosphorylation and decreased procaspase-6 cleavage in Flox but not in LAKO mice (FIGS. 15D and 15E). Of note, AMPK deficiency itself decreased procaspase-6 phosphorylation and increased procaspase-6 cleavage (FIGS. 15D and 15E). Moreover, CD-HFD decreased procaspase-6 phosphorylation, correlating with the increased procaspase-6 cleavage and decreased AMPK phosphorylation (FIG. 5K; FIG. 1B; and FIG. 15F).

### Example 7

### Caspase-6 Mediates a Feedforward Loop to Sustain the Caspase Cascade

To understand how caspase-6 controls the pathogenesis of NASH, its role in the apoptotic pathways were investigated. Depletion of caspase-6 in HepG2 cells significantly increased cell viability after 20 hrs of treatment with TNFα and CHX (FIG. 16A). An in vitro cleavage assay showed that procaspase-6 was directly cleaved by caspase-3 and -7, but not caspase-8 or -9 (FIG. 6A). Pre-treatment with a caspase-3 and -7 inhibitor largely attenuated procaspase-6 cleavage caused by TNFα and CHX (FIG. 6B). These data suggest that caspase-6 is activated by executioner caspases, but not initiators.

A relevant substrate was searched for and it was found that active caspase-6 cleaved purified Bcl2 family protein Bid (BH3 interacting-domain death agonist), but not Bax (Bcl2 associated X) in vitro (FIG. 6C). Both of these proteins contribute to cytochrome c release and subsequent cell damage (24, 25). Active caspase-6 cleaves Bid to generate two cleaved peptide fragments (FIG. 6D), one with a size similar to caspase-8-cleaved Bid (26), and another that was smaller N-terminal sequencing showed that active caspase-6 cleaved Bid at both Asp⁵⁹ and Asp⁷⁵ (FIGS. 6E and 6F); both cleavages activate Bid to induce cytochrome c release (26, 27). Because cleavage of Bid induces mitochondrial cytochrome c release into the cytoplasm (24, 25), livers from vehicle or VEID-treated (“VEID” disclosed as SEQ ID NO: 71) Flox and LAKO mice were fractionated and isolate cytosolic (Cyto) and mitochondrial (Mito) fractions were isolated. Cytosolic cytochrome c was significantly increased in LAKO mice. VEID (SEQ ID NO: 71) treatment decreased cytosolic cytochrome c in both Flox and LAKO mice, and completely abrogated the effect of LAKO (FIG. 6G).

To investigate whether caspase-6 might mediate a feedforward loop of the caspase cascade, because it is cleaved by the executioner caspases-3 and -7, and in turn induces cytochrome c release. HepG2 cells were transfected with scrambled control or caspase-6 siRNA and treated with vehicle or CHX plus TNFα for 2 hrs to induce caspase activation. 2 hrs after the medium change, amounts of cleaved caspase-9, -3 and -7 were similar in control or caspase-6 depleted cells. However, after 5 hrs, caspase-6 depleted cells had significantly less cleaved caspase-9, -3 and -7 (FIG. 6H; FIG. 16B). Thus, activation of the caspase cascade appears to diminish faster in caspase-6 depleted cells. Inhibition of caspase-6 with VEID (SEQ ID NO: 71) also led to a significant decrease of cleaved caspase-3 and -7 in primary hepatocytes (FIG. 16C). Caspase-6 may mediate a feedforward loop to sustain activation of the caspase cascade, in which cytochrome c can potentiate the activation of the upstream caspases, and this sustained activation may be necessary for extensive apoptosis (FIG. 6I). Importantly, this process is only activated under conditions of excess energy accumulation due to reduced AMPK activity.

### REFERENCES

References are cited herein throughout using the format of reference number(s) enclosed by parentheses corresponding to one or more of the following numbered references. For example, citation of references numbers 1 and 2 immediately herein below would be indicated in the disclosure as (1, 2).

1. V. Hernandez-Gea, S. L. Friedman, Pathogenesis of liver fibrosis. Annu Rev Pathol 6, 425-456 (2011).

2. S. L. Friedman, B. A. Neuschwander-Tetri, M. Rinella, A. J. Sanyal, Mechanisms of NAFLD development and therapeutic strategies. Nat Med 24, 908-922 (2018).

3. G. K. Michalopoulos, M. DeFrances, Liver regeneration. Adv Biochem Eng Biotechnol 93, 101-134 (2005).

4. M. E. Guicciardi, G. J. Gores, Apoptosis: a mechanism of acute and chronic liver injury. Gut 54, 1024-1033 (2005).

5. T. Luedde, N. Kaplowitz, R. F. Schwabe, Cell death and cell death responses in liver disease: mechanisms and clinical relevance. Gastroenterology 147, 765-783 e764 (2014).

6. D. G. Hardie, AMP-activated protein kinase: an energy sensor that regulates all aspects of cell function. Genes Dev 25, 1895-1908 (2011).

7. D. G. Hardie, F. A. Ross, S. A. Hawley, AMPK: a nutrient and energy sensor that maintains energy homeostasis. Nat Rev Mol Cell Biol 13, 251-262 (2012).

8. T. L. Martin et al., Diet-induced obesity alters AMP kinase activity in hypothalamus and skeletal muscle. J Biol Chem 281, 18933-18941 (2006).

9. B. Viollet et al., AMPK inhibition in health and disease. Crit Rev Biochem Mol Biol 45, 276-295 (2010).

10. R. J. Valentine, K. A. Coughlan, N. B. Ruderman, A. K. Saha, Insulin inhibits AMPK activity and phosphorylates AMPK Ser(485/491) through Akt in hepatocytes, myotubes and incubated rat skeletal muscle. Archives of Biochemistry and Biophysics 562, 62-69 (2014).

11. P. Zhao et al., TBK1 at the Crossroads of Inflammation and Energy Homeostasis in Adipose Tissue. Cell 172, 731-743 (2018).

12. G. R. Steinberg et al., Tumor necrosis factor alpha-induced skeletal muscle insulin resistance involves suppression of AMP-kinase signaling. Cell Metabolism 4, 465-474 (2006).

13. N. Boudaba et al., AMPK Re-Activation Suppresses Hepatic Steatosis but its Downregulation Does Not Promote Fatty Liver Development. Ebiomedicine 28, 194-209 (2018).

14. D. R. McIlwain, T. Berger, T. W. Mak, Caspase Functions in Cell Death and Disease. Csh Perspect Biol 5, (2013).

15. I. Budihardjo, H. Oliver, M. Lutter, X. Luo, X. D. Wang, Biochemical pathways of caspase activation during apoptosis. Annu Rev Cell Dev Bi 15, 269-290 (1999).

16. M. Matsumoto et al., An improved mouse model that rapidly develops fibrosis in non-alcoholic steatohepatitis. Int J Exp Pathol 94, 93-103 (2013).

17. J. R. Clapper et al., Diet-induced mouse model of fatty liver disease and nonalcoholic steatohepatitis reflecting clinical disease progression and methods of assessment. Am J Physiol Gastrointest Liver Physiol 305, G483-495 (2013).

18. M. Fujii et al., A murine model for non-alcoholic steatohepatitis showing evidence of association between diabetes and hepatocellular carcinoma. Med Mol Morphol 46, 141-152 (2013).

19. J. Y. Kim et al., ER Stress Drives Lipogenesis and Steatohepatitis via Caspase-2 Activation of S1P. Cell 175, 133-145 e115 (2018).

20. E. C. Cokorinos et al., Activation of Skeletal Muscle AMPK Promotes Glucose Disposal and Glucose Lowering in Non-human Primates and Mice. Cell Metab 25, 1147-1159 e1110 (2017).

21. D. Wu et al., Glucose-regulated phosphorylation of TET2 by AMPK reveals a pathway linking diabetes to cancer. Nature 559, 637-641 (2018).

22. A. Suzuki et al., Regulation of caspase-6 and FLIP by the AMPK family member ARK5. Oncogene 23, 7067-7075 (2004).

23. E. M. Velazquez-Delgado, J. A. Hardy, Phosphorylation regulates assembly of the caspase-6 substrate-binding groove. Structure 20, 742-751 (2012).

24. R. Eskes, S. Desagher, B. Antonsson, J. C. Martinou, Bid induces the oligomerization and insertion of Bax into the outer mitochondrial membrane. Mol Cell Biol 20, 929-935 (2000).

25. M. C. Wei et al., tBID, a membrane-targeted death ligand, oligomerizes BAK to release cytochrome c. Genes Dev 14, 2060-2071 (2000).

26. H. Li, H. Zhu, C. J. Xu, J. Yuan, Cleavage of BID by caspase 8 mediates the mitochondrial damage in the Fas pathway of apoptosis. Cell 94, 491-501 (1998).

27. V. R. Sutton et al., Initiation of apoptosis by granzyme B requires direct cleavage of bid, but not direct granzyme B-mediated caspase activation. J Exp Med 192, 1403-1414 (2000).

28. L. Drew, Fighting the fatty liver. Nature 550, S102-S103 (2017).

29. M. Hatting et al., Hepatocyte caspase-8 is an essential modulator of steatohepatitis in rodents. Hepatology 57, 2189-2201 (2013).

30. S. Thapaliya et al., Caspase 3 inactivation protects against hepatic cell death and ameliorates fibrogenesis in a diet-induced NASH model. Dig Dis Sci 59, 1197-1206 (2014).

31. W. J. Kaiser et al., RIP3 mediates the embryonic lethality of caspase-8-deficient mice. Nature 471, 368-372 (2011).

32. K. Kuida et al., Decreased apoptosis in the brain and premature lethality in CPP32-deficient mice. Nature 384, 368-372 (1996).

33. A. da Silva Morais et al., Development of hepatic fibrosis occurs normally in AMPK-deficient mice. Clin Sci (Lond) 118, 411-420 (2009).

34. D. E. Ehrnhoefer et al., A quantitative method for the specific assessment of caspase-6 activity in cell culture. PLoS One 6, e27680 (2011).

35. C. E. Heise et al., Mechanistic and structural understanding of uncompetitive inhibitors of caspase-6. PLoS One 7, e50864 (2012).

36. S. N. Shelton, M. E. Shawgo, J. D. Robertson, Cleavage of Bid by executioner caspases mediates feed forward amplification of mitochondrial outer membrane permeabilization during genotoxic stress-induced apoptosis in Jurkat cells. J Biol Chem 284, 11247-11255 (2009).

37. S. Ruchaud et al., Caspase-6 gene disruption reveals a requirement for lamin A cleavage in apoptotic chromatin condensation. EMBO J 21, 1967-1977 (2002).

38. A. C. LeBlanc, Caspase-6 as a novel early target in the treatment of Alzheimer's disease. Eur J Neurosci 37, 2005-2018 (2013).

39. X. J. Wang, Q. Cao, Y. Zhang, X. D. Su, Activation and Regulation of Caspase-6 and Its Role in Neurodegenerative Diseases. Annu Rev Pharmacol 55, 553-572 (2015).

40. A Salminen, K Kaarniranta, A. Haapasalo, H. Soininen, M. Hiltunen, AMP-activated protein kinase: a potential player in Alzheimer's disease. J Neurochem 118, 460-474 (2011).

41. J. Shi et al., Cleavage of GSDMD by inflammatory caspases determines pyroptotic cell death. Nature 526, 660-665 (2015).

The preceding Examples are offered for illustrative purposes only, and are not intended to limit the scope of the present invention in any way. Various modifications of the invention in addition to those shown and described herein will become apparent to those skilled in the art from the foregoing description and fall within the scope of the appended claims.

1. A method of treating or preventing a liver disease in a patient in need thereof, comprising administering to the patient a therapeutically effective amount of a therapeutictherapeutic agent that targets AM PK/Caspase-6 axis.

2. The method of claim 1, wherein the therapeutic agent inhibits caspase-6 activity.

3. The method of claim 1, wherein the therapeutic agent is Z-VEID-FMK (ZVF). (“VEID” disclosed as SEQ ID NO: 71).

4. The method of claim 1, wherein the therapeutic agent is an AMPK agonist that activates AMPK.

5. The method of claim 1, wherein the AMPK agonist is A-769662.

6. The method of claim 1, wherein the liver disease is nonalcoholic steatohepatitis (NASH).

7. A pharmaceutical composition for treating or preventing a liver disease in a patient in need thereof, comprising a therapeutically effective amount of one or more therapeutic agent that targets AMPK/Caspase-6 axis to inhibit caspase-6 activity, a pharmaceutically acceptable salt thereof, and a pharmaceutically acceptable carrier.

8. The pharmaceutical composition of claim 7, wherein the therapeutic agent inhibits caspase-6 activity.

9. The pharmaceutical composition of claim 7, wherein the caspase-6 inhibitor is Z-VEID-FMK (ZVF). (“VEID” disclosed as SEQ ID NO: 71)

10. The pharmaceutical composition of claim 7, wherein the therapeutic agent is an AMPK agonist that activates AMPK.

11. The pharmaceutical composition of claim 7, wherein the AMPK agonist is A-769662.

12. The pharmaceutical composition of claim 7, wherein each therapeutic agent is formulated in a pharmaceutically acceptable carrier or excipients for a proper administration, alone or in combination.

13. The pharmaceutical composition of claim 7, further comprising one or more other pharmaceutical therapeutic agent for treating or preventing a liver disease.

14. The pharmaceutical composition of claim 7, wherein the liver disease is nonalcoholic steatohepatitis (NASH).

15. A kit comprising
a) at least one therapeutic agent that targets AMPK/Caspase-6 axis; and
b) instructions for treating or preventing a liver disease in a patient in need thereof.

16. The kit of claim 15, wherein the least one therapeutic agent that targets AMPK/Caspase-6 axis comprises at least one therapeutic agent that inhibits caspase-6 activity.

17. The kit of claim 15, wherein the least one therapeutic agent that targets AMPK/Caspase-6 axis comprises Z-VEID-FMK (ZVF). (“VEID” disclosed as SEQ ID NO: 71).

18. The kit of claim 15, wherein the least one therapeutic agent that targets AMPK/Caspase-6 axis comprises at least one AMPK agonist.

19. The kit of claim 15, wherein the least one therapeutic agent that targets AMPK/Caspase-6 axis comprises at least one therapeutic agent that inhibits caspase-6 activity; Z-VEID-FMK (ZVF). (“VEID” disclosed as SEQ ID NO: 71); at least one AMPK agonist; or combinations thereof.

20. The kit of claim 19, wherein the least one therapeutic agent that targets AMPK/Caspase-6 axis comprises two or more therapeutic agents selected from at least one therapeutic agent that inhibits caspase-6 activity; Z-VEID-FMK (ZVF). (“VEID” disclosed as SEQ ID NO: 71); and at least one AMPK agonist.

21.-22. (canceled)


SEQUENCE LISTING (71 sequences)

SEQ ID NO: 1 - DNA, 21 bp, Artificial Sequence
  Description of Artificial Sequence Synthetic primer
gtcccagacatcagggagtaa

SEQ ID NO: 2 - DNA, 21 bp, Artificial Sequence
  Description of Artificial Sequence Synthetic primer
tcggatacttcagcgtcagga

SEQ ID NO: 3 - DNA, 21 bp, Artificial Sequence
  Description of Artificial Sequence Synthetic primer
ccccagtgtccttacagagtg

SEQ ID NO: 4 - DNA, 19 bp, Artificial Sequence
  Description of Artificial Sequence Synthetic primer
gtgcccagagtggatgtct

SEQ ID NO: 5 - DNA, 23 bp, Artificial Sequence
  Description of Artificial Sequence Synthetic primer
tggtgatgaaggggtcatttatg

SEQ ID NO: 6 - DNA, 21 bp, Artificial Sequence
  Description of Artificial Sequence Synthetic primer
ttcggctttccagtcagactc

SEQ ID NO: 7 - DNA, 20 bp, Artificial Sequence
  Description of Artificial Sequence Synthetic primer
ggaagtgttcgatccagccg

SEQ ID NO: 8 - DNA, 20 bp, Artificial Sequence
  Description of Artificial Sequence Synthetic primer
ggagggtcaggtgccaaaag

SEQ ID NO: 9 - DNA, 21 bp, Artificial Sequence
  Description of Artificial Sequence Synthetic primer
tgcttggactacatcccacac

SEQ ID NO: 10 - DNA, 21 bp, Artificial Sequence
  Description of Artificial Sequence Synthetic primer
tgcagtctaggaagttgacca

SEQ ID NO: 11 - DNA, 19 bp, Artificial Sequence
  Description of Artificial Sequence Synthetic primer
gctcctcttaggggccact

SEQ ID NO: 12 - DNA, 19 bp, Artificial Sequence
  Description of Artificial Sequence Synthetic primer
ccacgtctcaccattgggg

SEQ ID NO: 13 - DNA, 23 bp, Artificial Sequence
  Description of Artificial Sequence Synthetic primer
ctgtaacatggaaactggggaaa

SEQ ID NO: 14 - DNA, 22 bp, Artificial Sequence
  Description of Artificial Sequence Synthetic primer
ccatagctgaactgaaaaccac

SEQ ID NO: 15 - DNA, 18 bp, Artificial Sequence
  Description of Artificial Sequence Synthetic primer
ctccgcctgagccatgaa

SEQ ID NO: 16 - DNA, 21 bp, Artificial Sequence
  Description of Artificial Sequence Synthetic primer
caccagtgatgatgccattct

SEQ ID NO: 17 - DNA, 23 bp, Artificial Sequence
  Description of Artificial Sequence Synthetic primer
ttaaaaacctggatcggaaccaa

SEQ ID NO: 18 - DNA, 23 bp, Artificial Sequence
  Description of Artificial Sequence Synthetic primer
gcattagcttcagatttacgggt

SEQ ID NO: 19 - DNA, 23 bp, Artificial Sequence
  Description of Artificial Sequence Synthetic primer
atccacggcatactatcaacatc

SEQ ID NO: 20 - DNA, 21 bp, Artificial Sequence
  Description of Artificial Sequence Synthetic primer
caaggctcaccatcatcgtag

SEQ ID NO: 21 - DNA, 21 bp, Artificial Sequence
  Description of Artificial Sequence Synthetic primer
atcacagcctcaagtcagtgg

SEQ ID NO: 22 - DNA, 20 bp, Artificial Sequence
  Description of Artificial Sequence Synthetic primer
ttcaggtcatcgggttgcac

SEQ ID NO: 23 - DNA, 21 bp, Artificial Sequence
  Description of Artificial Sequence Synthetic primer
ggaggtggtgatagccggtat

SEQ ID NO: 24 - DNA, 21 bp, Artificial Sequence
  Description of Artificial Sequence Synthetic primer
tgggtaatccatagagcccag

SEQ ID NO: 25 - DNA, 21 bp, Artificial Sequence
  Description of Artificial Sequence Synthetic primer
cgactcgctatctccaagtga

SEQ ID NO: 26 - DNA, 20 bp, Artificial Sequence
  Description of Artificial Sequence Synthetic primer
gttgaaccagtctccgacca

SEQ ID NO: 27 - DNA, 22 bp, Artificial Sequence
  Description of Artificial Sequence Synthetic primer
gcaactgttcctgaactcaact

SEQ ID NO: 28 - DNA, 21 bp, Artificial Sequence
  Description of Artificial Sequence Synthetic primer
atcttttggggtccgtcaact

SEQ ID NO: 29 - DNA, 20 bp, Artificial Sequence
  Description of Artificial Sequence Synthetic primer
acacacacacatgctcacac

SEQ ID NO: 30 - DNA, 20 bp, Artificial Sequence
  Description of Artificial Sequence Synthetic primer
atcaccgcatagtctctgaa

SEQ ID NO: 31 - DNA, 19 bp, Artificial Sequence
  Description of Artificial Sequence Synthetic primer
gaggaagccgagatacccc

SEQ ID NO: 32 - DNA, 21 bp, Artificial Sequence
  Description of Artificial Sequence Synthetic primer
tgctgtggatctgacttcgag

SEQ ID NO: 33 - DNA, 22 bp, Artificial Sequence
  Description of Artificial Sequence Synthetic primer
catccgctcctttgatgatctt

SEQ ID NO: 34 - DNA, 21 bp, Artificial Sequence
  Description of Artificial Sequence Synthetic primer
gtgctcgggtcatgttcaagt

SEQ ID NO: 35 - DNA, 21 bp, Artificial Sequence
  Description of Artificial Sequence Synthetic primer
tccatgctagactcagaagtc

SEQ ID NO: 36 - DNA, 21 bp, Artificial Sequence
  Description of Artificial Sequence Synthetic primer
tcccggtggacacaatttttc

SEQ ID NO: 37 - DNA, 24 bp, Artificial Sequence
  Description of Artificial Sequence Synthetic primer
gagaatgaggcaaacttgctagcg

SEQ ID NO: 38 - DNA, 24 bp, Artificial Sequence
  Description of Artificial Sequence Synthetic primer
tgcatggttctgagtgctaagacc

SEQ ID NO: 39 - DNA, 20 bp, Artificial Sequence
  Description of Artificial Sequence Synthetic primer
ccactgtcgcttttcgccgc

SEQ ID NO: 40 - DNA, 23 bp, Artificial Sequence
  Description of Artificial Sequence Synthetic primer
tgcaaacagctcgaaggagacgc

SEQ ID NO: 41 - DNA, 22 bp, Artificial Sequence
  Description of Artificial Sequence Synthetic primer
tactcaaccggcagaagattcg

SEQ ID NO: 42 - DNA, 21 bp, Artificial Sequence
  Description of Artificial Sequence Synthetic primer
tccttttcgtccaaccttcca

SEQ ID NO: 43 - DNA, 21 bp, Artificial Sequence
  Description of Artificial Sequence Synthetic primer
gacttccttcacagcctcatc

SEQ ID NO: 44 - DNA, 23 bp, Artificial Sequence
  Description of Artificial Sequence Synthetic primer
cgagcgactatcaaagacatacg

SEQ ID NO: 45 - DNA, 21 bp, Artificial Sequence
  Description of Artificial Sequence Synthetic primer
gaagacagacctagacagcgg

SEQ ID NO: 46 - DNA, 21 bp, Artificial Sequence
  Description of Artificial Sequence Synthetic primer
ccagtagcttcaccactcgac

SEQ ID NO: 47 - DNA, 23 bp, Artificial Sequence
  Description of Artificial Sequence Synthetic primer
tctgtcaagttatggcctactgg

SEQ ID NO: 48 - DNA, 19 bp, Artificial Sequence
  Description of Artificial Sequence Synthetic primer
ggaacacgactccgaaccc

SEQ ID NO: 49 - DNA, 19 bp, Artificial Sequence
  Description of Artificial Sequence Synthetic primer
tgacccggctattccgtga

SEQ ID NO: 50 - DNA, 21 bp, Artificial Sequence
  Description of Artificial Sequence Synthetic primer
ctgggctgagcaatacagttc

SEQ ID NO: 51 - DNA, 19 bp, Artificial Sequence
  Description of Artificial Sequence Synthetic primer
ctcccgtggcttctagtgc

SEQ ID NO: 52 - DNA, 22 bp, Artificial Sequence
  Description of Artificial Sequence Synthetic primer
gccttagtttggacaggatctg

SEQ ID NO: 53 - DNA, 21 bp, Artificial Sequence
  Description of Artificial Sequence Synthetic primer
gcaactcggacctggtcataa

SEQ ID NO: 54 - DNA, 19 bp, Artificial Sequence
  Description of Artificial Sequence Synthetic primer
cggcccgtgatgagaaact

SEQ ID NO: 55 - DNA, 23 bp, Artificial Sequence
  Description of Artificial Sequence Synthetic primer
ccctcacactcagatcatcttct

SEQ ID NO: 56 - DNA, 18 bp, Artificial Sequence
  Description of Artificial Sequence Synthetic primer
gctacgacgtgggctaca

SEQ ID NO: 57 - protein, 10 aa, Unknown
  Description of Unknown AMPK substrate motif
MRRASSYSSL

SEQ ID NO: 58 - protein, 10 aa, Unknown
  Description of Unknown AMPK substrate motif
LSKSSSSPEL

SEQ ID NO: 59 - protein, 10 aa, Unknown
  Description of Unknown AMPK substrate motif
IRSSMSGLHL

SEQ ID NO: 60 - protein, 10 aa, Unknown
  Description of Unknown AMPK substrate motif
IRRSGSTSPL

SEQ ID NO: 61 - protein, 10 aa, Unknown
  Description of Unknown AMPK substrate motif
LPSSPSSATP

SEQ ID NO: 62 - protein, 10 aa, Unknown
  Description of Unknown AMPK substrate motif
IKRTVSEPSL

SEQ ID NO: 63 - protein, 10 aa, Unknown
  Description of Unknown AMPK substrate motif
VNRKVSQRRV

SEQ ID NO: 64 - protein, 57 aa, Homo sapiens
ETVNGSWYIQDLCEMLGKYGSSLEFTELLTLVNRKVSQRRVDFCKDPSAIGKKQVPC

SEQ ID NO: 65 - protein, 57 aa, Mus sp.
ETVNGSWYIQDLCEMLARYGSSLEFTELLTLVNRKVSQRRVDFCKDPDAIGKKQVPC

SEQ ID NO: 66 - protein, 57 aa, Rattus sp.
ETVNGSWYIQDLSEMLARHGSSLEFTELLTLVNRKVSQRRVDFCKDPGAIGKKQVPC

SEQ ID NO: 67 - protein, 57 aa, Bos sp.
ETVNGSWYIQDLCEMLGKFGSSLEFTELLTLVNRKVSQRRVDFCRDPNAIGKKQVPC

SEQ ID NO: 68 - protein, 6 aa, Artificial Sequence
  Description of Artificial Sequence Synthetic peptide
GNRSSH

SEQ ID NO: 69 - protein, 6 aa, Artificial Sequence
  Description of Artificial Sequence Synthetic peptide
SESQED

SEQ ID NO: 70 - protein, 194 aa, Artificial Sequence
  Description of Artificial Sequence Synthetic polypeptide
MDCEVNNGSSLRDECITNLLVFGFLQSCSDNSFRRELDALGHELPVLAPQWEGYDELQTDGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTSLLRDVFHTTVNFINQNLRTYVRSLARNGMD

SEQ ID NO: 71 - protein, 4 aa, Artificial Sequence
  Description of Artificial Sequence Synthetic peptide
VEID